(19) United States
(12) Patent Application Publication
(10) Pub. No.: US 2025/0041394 A1
(43) Pub. Date: Feb. 6, 2025

(54) IMMUNOSTIMULATORY MRNA COMPOSITIONS AND USES THEREOF

(71) Applicant: eTheRNA immunotherapies NV, Niel (BE)

(72) Inventors: Florence Lambolez, Niel (BE); Elisabeth Brabants, Niel (BE); Jessica Filtjens, Niel (BE); Stefaan De Koker, Niel (BE)

(73) Assignee: eTheRNA immunotherapies NV, Niel (BE)

(21) Appl. No.: 18/718,199

(22) PCT Filed: Dec. 22, 2022
(86) PCT No.: PCT/EP22/87434

§ 371 Date: Jun. 10, 2024

(30) Foreign Application Priority Data
Dec. 23, 2021 (EP) 21217374.4

(51) Int. Cl. A61K 39/00; A61K 39/39; A61K 39/395; A61P 35/00

(52) U.S. Cl. CPC A61K 39/0011; A61K 39/39; A61K 39/3955; A61P 35/00; A61K 2039/53; A61K 2039/55516; A61K 2039/55522; A61K 2039/55527; A61K 2039/55538

(57) ABSTRACT

The invention is situated in the field of immunotherapy. More specifically, the invention is related to a composition of one or more nucleic acid molecule(s) encoding functional immunostimulatory proteins such as IL-21 and encoding a co-stimulatory molecule, optionally in combination with a cytokine, and/or a chemokine to enhance the host immune response against tumor cells present in the body. The invention further relates to nucleic acid containing compositions or pharmaceutical formulations for use in the treatment of patients suffering from a tumor and/or cancer disease.

### FIELD OF THE INVENTION

The invention is situated in the field of immunotherapy. More specifically, the invention is related to a composition of one or more nucleic acid molecule(s) encoding functional immunostimulatory proteins such as IL-21 and a co-stimulatory molecule, optionally in combination with a cytokine, and/or a chemokine to enhance the host immune response against tumor cells present in the body. The invention further relates to nucleic acid containing compositions or pharmaceutical formulations for use in the treatment of patients suffering from a tumor and/or cancer disease.

### BACKGROUND OF THE INVENTION

Immune-therapies aimed at activating immune responses against tumor cells have revolutionized cancer treatment and are providing clinical benefit to previously untreatable patients. Long term benefit of immune-therapies requires the induction, expansion and maintenance of memory T cells that recognize cancer associated antigens. Systemic delivery of checkpoint inhibitors (CPI)-antibodies blocking T cell inhibitory receptors such as CTLA-4 and PD-1/PD-L1—has resulted in durable clinical responses by re-activating/expanding pre-existing T cells, but many tumors have evolved immune-suppressive strategies making them CPI resistant, requiring the development of complementary strategies resulting in enhanced T cell immunity.

Induction of optimal T cell responses requires antigen presentation to the T cell receptor (TCR) in combination with additional T cell activating signals mediated by co-stimulatory ligands and cytokines or chemokines. Nucleic acid-based approaches including viral vectors, plasmid DNA and messenger mRNA have been used to locally encode these immune-stimulatory proteins in the tumor-micro-environment, resulting in the induction and amplification of systemic immune responses against the antigens present in the tumor bed. These nucleic acids encoding immune-stimulatory proteins can also be used to promote the generation of T cell responses against co-delivered antigens in a vaccination context.

Although individual immune-stimulatory proteins can elicit some level of anti-tumor efficacy, multiple immune-stimulatory pathways need to be targeted simultaneously to enhance therapeutic benefit. Which factors to combine to yield optimal efficacy however remains a huge challenge, which is the topic of this invention.

### SUMMARY OF THE INVENTION

The inventors have established that the (local) delivery of a composition comprising nucleic acids encoding at least the functional immunostimulatory protein IL-21; induces systemic immune responses leading to tumor reduction or tumor growth delay.

Moreover, combination of said composition comprising nucleic acids encoding the functional immunostimulatory protein IL-21, with one or more of a co-stimulatory molecule, a cytokine, and/or a chemokine; further boosts the observed effects.

The invention is specifically directed to a composition comprising one or more isolated non-viral nucleic acid molecule(s) encoding the functional immunostimulatory protein IL-21 and encoding a co-stimulatory molecule. In a specific embodiment, said composition may further comprise one or more nucleic acid molecules encoding a cytokine and/or a chemokine.

The invention provides the proof of concept that such compositions can induce/enhance an anti-tumoral effect by amongst others reducing tumor volume after delivery and thus forms a promising new approach for anti-tumor immunotherapy.

The invention thus provides solutions for improving the immunostimulatory characteristics of compositions comprising the introduction of at least one nucleic acid, in particular mRNA or DNA molecule, encoding proteins that modify the tumour microenvironment characterized in that amongst the encoded functional immunostimulatory protein is at least IL-21, optionally in combination with one or more of a co-stimulatory molecule, a cytokine, and/or a chemokine

In a specific embodiment, the composition comprises a nucleic acid molecule encoding an IL-21 isoform, in particular an IL-21 isoform selected from the list comprising IL-21 isoform 1 and IL-21 isoform 2, more in particular IL-21 isoform 1.

In a particular embodiment, the composition further comprises one or more of: a target-specific antigen, and/or a checkpoint inhibitor.

In some embodiments, the composition comprises a co-stimulatory molecule selected from the list comprising 4-1BBL, OX40L, ICOS ligand and CD40L.

In a particular embodiment, the composition comprises a cytokine selected from the list comprising IL-7, IL-12, IL-18, IFNlambda, IL-15sushi IL-23, a type I IFN, a type II IFN, a type III IFN.

In another embodiment, the composition comprises a chemokine selected from the list comprising CCL19, CCL20, CCL21, CXCL9, CXCL10, CCL4, CCL5 and XCL1.

In a specific embodiment, the composition comprises a target-specific antigen such as a tumor-associated or tumor-specific antigen or a neoantigen.

Said target-specific antigen can be derived from either one of: total mRNA isolated from (a) target cell(s), one or more specific target mRNA molecules, protein lysates of (a) target cell(s), specific proteins from (a) target cell(s), or a synthetic target-specific peptide or protein and synthetic mRNA or DNA encoding a target-specific antigen or its derived peptides.

In a particular embodiment, the composition of the present invention comprises a checkpoint inhibitor, such as a checkpoint inhibitor polypeptide in particular selected from the list comprising inhibitory molecules of: PD-1, PD-L1, CTLA4, TIGIT, TIM3, LAG-3 and VISTA; preferably inhibitory molecules of PD-1, PD-L1, or CTLA4.

A preferred combination of immunostimulatory factors used in the methods of the invention is IL-21 and 4-1BBL. In other preferred embodiments, the combination of IL-21, 4-1BBL and IL-7 immunostimulatory molecules is used. In yet other preferred embodiments, the combination of IL-21, 4-1BBL and IFNlambda immunostimulatory molecules; or the combination of IL-21, 4-1BBL and IL-18 is used. In other preferred embodiments, the combination of IL-21, 4-1BBL and IL-15 is used. In other preferred embodiments, the combination of IL-21, 4-1BBL and IL-15sushi is used.

In a particular embodiment, the mRNA or DNA molecules are separate nucleic acid molecules. In another embodiment, the mRNA or DNA molecules are part of one single nucleic acid molecule, wherein the single nucleic acid molecule is capable of expressing the two or more immunostimulatory proteins simultaneously e.g. the two or more mRNA or DNA molecules encoding the immunostimulatory proteins may be linked in the single mRNA or DNA molecule by an internal ribosomal entry site (IRES) or a self-cleaving 2a peptide encoding sequence.

In certain embodiments, the nucleic acid molecule in the composition is selected from the group comprising RNA, DNA, preferably RNA, more preferably mRNA.

In a particular embodiment, the nucleic acid molecule of the composition comprises one or more of the following: a 5′CAP, a poly(A) tail and/or modified nucleoside(s); wherein said modified nucleoside may be N1-methylpseudouridine.

In a specific embodiment, the nucleic acid molecules of the composition are in the form of naked nucleic acid molecules or nucleic acids encapsulated in nanoparticles, such as lipid nanoparticles or polymeric nanoparticle, in particular lipid nanoparticles.

The invention further provides a lipid nanoparticle comprising the composition as described by the invention.

In another embodiment, the composition or the lipid nanoparticle comprises an ionizable.lipid, cholesterol, a phospholipid and a PEGylated lipid.

In a preferred embodiment, the composition or the lipid nanoparticle comprises about and between 35 mol % and 65 mol % of ionizable lipid; about and between 5 mol % and 25 mol % of phospholipid; about and between 0.5 mol % and 3.0 mol % of PEG lipid; balanced by an amount of sterol; in particular 50 mol % of ionizable lipid, 10 mol % of phospholipid, 1.5 mol % of PEG lipid and 38.5 mol % of sterol.

The invention further provides a pharmaceutical formulation comprising the composition or the lipid nanoparticle of the invention and at least one pharmaceutically acceptable carrier or excipient. Pharmaceutical formulations of the invention are particularly suitable as a vaccine.

In a further aspect, the present invention provides the composition, the lipid nanoparticle or the pharmaceutical formulation as defined herein for use in parenteral administration; more in particular for use in intravenous, intratumoral, intradermal, intraperitoneal, intramuscular or intranodal administration, preferably intratumoral administration.

In a particular embodiment, the composition, the lipid nanoparticle or the pharmaceutical formulation of the invention is provided for use in human or veterinary medicine.

In a preferred embodiment, the composition, the lipid nanoparticle or the pharmaceutical formulation as defined herein is provided for use in combination with standard of care cancer therapies including radiotherapy and chemotherapy.

In a more preferred embodiment, the composition, the lipid nanoparticle or the pharmaceutical formulation of the invention is provided for use in the prevention and/or treatment of cell proliferative disorders.

In yet another embodiment, the composition, the lipid nanoparticle or the pharmaceutical formulation as defined herein is provided for use in eliciting an immune response towards a tumor and/or cancer in a subject.

### BRIEF DESCRIPTION OF THE DRAWINGS

FIG. 1. mRNA monotherapy with IL-21, IL-7, IL-15sushi or 4-1BBL in a MC38 colon (colon cancer) model. The tumor volume (mm³) is shown for each time point when tumours were measured post-randomisation. Panel A shows treatment with Fluc mRNA control (3 times 15 μg mRNA/dose formulated in LNPs) at days 0, 3 and 7 post-randomisation while panel B, C, D, E shows treatment with mRNA encoding respectively IL-21 iso2, IL-7, 4-1BBL and IL-15sushi at the same dose and treatment regime. For the control and IL-15sushi, a complete response (“CR”) was observed in 2 of 8 subjects (20%) while treatment with the IL-21 mRNA elicited complete responses in 6 of 8 animals (75%). Monotherapy treatment with IL-7 or 4-1BBL showed no impact on tumour volume.

FIG. 2. Combination mRNA therapy with IL-7 and IL-21 in a MC38 (colon cancer) model. The tumor volume (mm³) is shown for each time point when tumours were measured post-randomisation. Panel A shows treatment with NanoLuc mRNA control (3 times 10 μg mRNA/dose formulated in LNPs) at days 0, 3 and 7 post-randomisation while panels B and C show the treatment with NanoLuc control mRNA with mRNA encoding respectively IL-21 or IL-7 (3 times at a reduced dose of 5 μg for each mRNA/dose). Panel D represents the combination therapy with mRNA encoding IL-7 and IL-21 (3 times at a dose of 5 μg for each mRNA/dose).

FIG. 3. Combination mRNA therapy with IL-15sushi and IL-21 in a MC38 (colon cancer) model. The tumor volume (mm³) is shown for each time point when tumours were measured post-randomisation. Panel A shows a monotherapy treatment with mRNA encoding IL-21 (3 times at 15 μg total mRNA/dose) while panel B shows a combination therapy with mRNAs encoding IL-21 and IL-15sushi mRNA (3×7.5 μg each mRNA/dose); panel C a combination therapy with mRNAs encoding IL-21, IL-7 and 4-1BBL mRNA (3×5 μg each mRNA/dose) and panel D a combination therapy with mRNAs encoding IL-21, 4-1BBL and IL-15 sushi (3×5 μg each mRNA/dose);

FIG. 4. Generation of systemic memory Immune Response after treatment with IL-21 monotherapy or IL-based mix therapy. The tumor volume (mm³) is shown for each time point when tumours were measured post-randomisation. The existence of memory immune response in animals treated with IL-21 monotherapy therapy (panel A and B) or combinational therapy (panel C and D) was evaluated. Panel A depicts tumor growth for naive animals (n=12) injected de novo with the MC238 cancer cell line. Panel B depicts tumor growth in rechallenged mice (n=6) previously treated with II-21 monotherapy; Panel C depicts tumor growth in rechallenged mice (n=6) previously treated with a combination therapy of IL-21/4-1BBL/IL-15sushi. Panel D depicts tumor growth in rechallenged mice (n=8) previously treated with a combination therapy of -21/4-1BBL/IL-7.

FIG. 5. Generation of systemic memory Immune Response after treatment with IL-21 monotherapy or IL-based mix therapy. The tumor volume (mm³) is shown for each time point when tumours were measured post-randomisation. Panel A depicts tumor growth for TBS, empty LNP and non-translated IL-21 mRNA LNP treated animals. Panel B depicts tumor growth for IL-21, IL-7 or 4-1BBL mRNA LNP treated animals. Panel C depicts tumor growth for animals treated with an IL-21/IL-7 mRNA LNP combination, IL-21/4-1BBL mRNA LNP combination or IL-7/4-1BBL mRNA LNP combination. Panel D depicts tumor growth for an IL-21/IL-7/4-1BBL mRNA LNP combination.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

In search for new methods to induce systemic antitumor immune responses, the inventors investigated whether local administration of a composition comprising LNP encapsulated nucleic acids encoding immunomodulatory molecules induces an effective anti-tumour response. The inventors unexpectedly found that a composition comprising nucleic acids encoding the functional immunostimulatory protein IL-21 resulted in strong and long-lasting anti-tumor responses upon direct intratumoral injection.

It was further established that combining IL-21mRNA with mRNA molecules encoding 4-1BBL and IL-7 further enhanced efficacy.

While the present invention is suited to activate and expand anti-tumor immunity by direct modulation of the tumor micro-environment upon intratumoral administration, it might also be suitable to enhance T cell responses to co-delivered antigens in a vaccination context (eg intramuscular, intranodal or intravenous immunization).

The term “target” used throughout the description is not limited to the specific examples that may be described herein. tumor or cancer cell may be targeted. The term “target-specific antigen” used throughout the description is not limited to the specific examples that may be described herein. It will be clear to the skilled person that the invention is related to enhancing anti-tumour responses by providing factors that modify the tumour microenvironment.

Without wanting to limit the scope of protection of the invention, some examples of possible markers are listed below.

The term “antigen presenting cell” used throughout the description includes all antigen presenting cells. Specific non limiting examples are dendritic cells, dendritic cell-lines, b-cells, or B-cell-lines. The dendritic cells or B-cells can be isolated or generated from the blood of a patient or healthy subject. The patient or subject can have been the subject of prior vaccination or not.

The terms “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma. Specific antigens for cancer can e.g. be MelanA/MART1, Cancer-germline antigens, gplOO, Tyrosinase, CEA, PSA, Her-2/neu, survivin, telomerase.

The invention thus provides a composition comprising a nucleic acid molecule encoding the functional immunostimulatory protein IL-21, and optionally encoding one or more of a co-stimulatory molecule, a cytokine, and/or a chemokine. In particular, the present invention provides a composition comprising one or more isolated non-viral nucleic acid molecule(s) encoding the functional immunostimulatory protein IL-21 and encoding a co-stimulatory molecule. Said composition may further optionally comprise one or more nucleic acid molecules encoding a cytokine and/or a chemokine.

In the context of the present invention, the term “immunostimulatory protein” is to be understood as any protein able to stimulate the immune system by inducing activation or increasing activity of any of its components. As used herein, said immunostimulatory proteins provide antigenic specificity in immune responses as a result of for example vaccine administration.

In the context of the present invention, the term “interleukin 21” is to be understood as a cytokine that has potent regulatory effects on cells of the immune system, including natural killer (NK) cells and cytotoxic T cells that can destroy virally infected or cancerous cells. IL-21 has anti-tumor effects through continued and increased CD8+ cell response to achieve enduring tumor immunity.

In a specific embodiment, the composition comprises a nucleic acid molecule encoding an IL-21 isoform, in particular an IL-21 isoform selected from the list comprising IL-21 isoform 1 and IL-21 isoform 2, more in particular IL-21 isoform 1.

In the context of the present invention, the term “co-stimulatory molecule” is to be understood as a heterogenous group of cell surface molecules that act to amplify or counteract the initial activating signals provided to T cells from the T cell receptor (TCR) following its interaction with an antigen/major histocompatibility complex (MHC), thereby influencing T cell differentiation and fate. As used herein, said co-stimulatory molecules may be one of the molecules in the class of the immunoglobulin (Ig) superfamily, the tumor necrosis factor (TNF)-TNF receptor (TNFR) superfamily and the T cell Ig and mucin (TIM) domain family.

In some embodiments, the co-stimulatory molecule is selected from the list comprising 4-1BBL, OX40L, ICOS ligand and CD40L.

In the context of the present invention, the term “cytokine” is to be understood as a protein important in cell signaling. As used herein, cytokines are immunomodulating cell signaling proteins and encompass interferons, interleukins, lymphokines, and tumor necrosis factors, but not hormones or growth factors. Cytokines act through cell surface receptors and are especially important in the immune system; cytokines modulate the balance between humoral and cell-based immune responses, and they regulate the maturation, growth, and responsiveness of particular cell populations. In particular, cytokines as defined in the present invention may be particularly selected from the list comprising: IL-7, IL-12, IL-18, IFNlambda, IL-15sushi, IL-23, a type I IFN, a type II IFN, a type III IFN.

In a particular embodiment, the cytokine is selected from the list comprising IL-7, IL-12, IL-18, IFNlambda, and IL-15sushi.

In a specific embodiment, the composition of the present invention comprises a nucleic acid molecule encoding an IL-21 isoform, and a cytokine IL-7 isoform, in particular an IL-7 isoform selected from the list comprising IL-7 isoform 1 and IL-7 isoform 2, more in particular IL-7 isoform 1.

In another specific embodiment, the composition of the present invention comprises a nucleic acid molecule encoding an IL-21 isoform 1, and an IL-7 isoform 1.

In yet another specific embodiment, the composition of the present invention comprises a nucleic acid molecule encoding an IL-21 isoform 1, and IL-15sushi.

In the context of the present invention, the term “chemokine” is to be understood as signaling proteins secreted by cells that induce directional movement of leukocytes, as well as other cell types, including endothelial and epithelial cells. Chemokines interact with G protein-linked transmembrane receptors. As used herein, said chemokine can be selected from the group comprising: CXC, CC, CX3C and C motifs.

In some embodiments, said chemokine can be selected from the list comprising: CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL1-V21C/V59C, XCL2 CX3CL1.

Delivering nucleic acids encoding an additional chemokine into the tumor environment could be beneficial, such as for example using chemokines that attract dendritic cells (e.g. CCL4, CCL5, CCL19, CCL20, CCL21, XCL1) or using T-cell/NK attracting chemokines (e.g. CCL3, CCL5, CXCL9-10_CCL19, XCL1)

In a preferred embodiment, the chemokine is selected from the list comprising CCL19, CCL20, CCL21, CXCL9, CXCL10, CCL4, CCL5 and XCL1.

In a particular embodiment, the composition further comprises one or more of: a target-specific antigen and/or, and/or a checkpoint inhibitor.

In a specific embodiment, the composition comprises a target-specific antigen being a tumor antigen. Said target-specific antigen can be provided in the form of either one of: total mRNA isolated from (a) target cell(s), one or more target-specific mRNA molecules, protein lysates of (a) target cell(s), specific proteins from (a) target cell(s), or a synthetic target-specific peptide or protein and synthetic mRNA or DNA encoding a target-specific antigen or its derived peptides.

In the context of the present invention, the term “checkpoint inhibitor” is to be understood as a type of drug or molecule that blocks immune checkpoints that are exploited by tumor cells to decrease immune activation and antigen recognition. Therefore, these checkpoint inhibitors can also be a powerful tool to improve cancer therapy.

In a particular embodiment, the composition comprises one or more nucleic acid molecules encoding for any compound able to directly or indirectly affect the regulation of said checkpoint inhibitor by reducing for example the expression of said checkpoint inhibitor (i.e., transcription and/or the translation) or its natural ligands, or the checkpoint inhibitor activity. Without being so limited, such inhibitors include proteins, peptides, small molecules, antibodies, etc. that block said checkpoint inhibitor-associated signaling molecule or pathway.

In a particular embodiment, the checkpoint inhibitor is selected from the list comprising: inhibitory molecules of: PD-1, PD-L1, CTLA4, TIGIT, TIM3, LAG-3 and VISTA; preferably inhibitory molecules of PD-1, PD-L1, or CTLA4.

For example, said composition may comprise anti-PD1 antibodies directed against PD-1, such as nivolumab (BMS-936558/MDX1106), pidilizumab (CT-011) or pembrolizumab (MK-3475). Alternatively, PD-L1 inhibitors such as atezolizumab or durvalumab may also be suitably used within the context of the invention.

Exemplary human anti-CTLA4 antibodies are described in detail in for example WO 00/37504. Such antibodies include, but are not limited to, 3.1.1, 4.1.1, 4.8.1, 4.10.2, 4.13.1, 4.14.3, 6.1.1, ticilimumab, 11.6.1, 11.7.1, 12.3.1.1, and 12.9.1.1, as well as tremelimumab and ipilimumab.

The above described anti-checkpoint inhibitor antibodies such as anti-PD-1 and/or anti-PD-L1/PD-L2 antibodies are well known in the art. Other blocking antibodies may be readily identified and prepared by the skilled person.

In a very specific embodiment, the present invention provides a composition comprising one or more isolated non-viral nucleic acid molecule(s) encoding the functional immunostimulatory protein IL-21 and optionally encoding one or more of:
a co-stimulatory molecule selected from the list comprising 4-1BBL, OX40L, ICOS ligand, and CD40L;
a cytokine selected from the list comprising IL-7, IL-12, IL-18, IFNlambda, IL-15sushi, IL-23, a type I IFN, a type II IFN, a type III IFN;
a chemokine selected from the list comprising CCL19, CCL20, CCL21, CXCL9, CXCL10, CCL4, CCL5 and XCL1;
an inhibitory molecule of: PD-1, PD-L1, CTLA4, TIGIT, TIM3, LAG-3 and/or VISTA; preferably inhibitory molecules of PD-1, PD-L1, or CTLA4.

In another very specific embodiment, the invention provides a composition comprising one or more isolated non-viral nucleic acid molecule(s) encoding the functional immunostimulatory protein IL-21 and optionally encoding one or more of IL-7, 4-1BBL, IFNlambda, IL-18, IL-15sushi and/or CCL5.

A preferred combination of immunostimulatory factors used in the methods of the invention is IL-21 and 4-1BBL. In other preferred embodiments, the combination of IL-21, 4-1BBL and IL-7 immunostimulatory molecules is used. In yet other preferred embodiments, the combination of IL-21, 4-1BBL and IL-15sushi immunostimulatory molecules is used.

In a specific embodiment, the composition comprises a nucleic acid molecule encoding an IL-21 isoform 1, and 4-1BBL.

In another specific embodiment, the composition of the present invention comprises a nucleic acid molecule encoding an IL-21 isoform 2, 4-1BBL, and IL-7 isoform 1.

In yet another specific embodiment, the composition of the present invention comprises a nucleic acid molecule encoding an IL-21 isoform 2, 4-1BBL, and IL-15sushi.

In yet another embodiment, the composition of the present invention comprises a nucleic acid molecule encoding an IL-21 isoform 2, 4-1BBL and IFNlambda

In yet another embodiment, the composition of the present invention comprises a nucleic acid molecule encoding an IL-21 isoform 2, 4-1BBL and IL-18.

The SEQ ID numbers for the human amino acid sequence for immunostimulatory proteins defined in the invention, are listed in Table 1.

It should be understood that the amino acid sequences described herein are not limitative and can encompass sequence variation (i.e. have a percentage sequence identity to the described sequence).

Hence, in a particular embodiment, the human amino acid sequences described herein are at least and/or about 90%, 91%, 92%, 93%, 94%, 95%, 96%, 97%, 98%, 99% or 100% identical to the SEQ IDs recited in Table 1.

The present invention may also provide a composition comprising nucleic acids encoding immunostimulatory proteins, co-stimulatory molecules, cytokines, and/or chemokines, wherein the human amino acid sequences are replaced by the respective mouse amino acid sequences. e.g. as presented in Table 2, such as those used in the examples part disclosed herein

Hence, in a particular embodiment, the mouse amino acid sequences described herein are at least and/or about 75%, 80%, 85%, 90%, 91%, 92%, 93%, 94%, 95%, 96%, 97%, 98%, 99% or 100% identical to the SEQ IDs recited in Table 2.

For the purposes of comparing two or more nucleotide or amino acid (AA) sequences, the percentage of “sequence identity” between a first nucleotide/AA sequence and a second nucleotide/AA sequence may be calculated by dividing [the number of nucleotides/AA in the first nucleotide/AA sequence that are identical to the nucleotides/AA at the corresponding positions in the second nucleotide/AA sequence] by [the total number of nucleotides/AA in the first nucleotide/AA sequence] and multiplying by [100%], in which each deletion, insertion, substitution or addition of a nucleotide/AA in the second nucleotide/AA sequence-compared to the first nucleotide/AA sequence—is considered as a difference at a single nucleotide/AA (position). Alternatively, the degree of sequence identity between two or more nucleotide/AA sequences may be calculated using a known computer algorithm for sequence alignment such as NCBI Blast v2.0, using standard settings. A specific method utilizes the BLAST module of WU-BLAST-2 set to the default parameters, with overlap span and overlap fraction set to 1 and 0.125, respectively.

Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged)amides: Asp, Asn, Glu and Gln; (c) polar, positively charged residues: His, Arg and Lys; (d) large aliphatic, nonpolar residues: Met, Leu, IIe, Val and Cys; and (e) aromatic residues: Phe, Tyr and Trp. Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; IIe into Leu or into Val; Leu into IIe or into Val; Lys into Arg, into Gln or into Glu; Met into Leu, into Tyr or into IIe; Phe into Met, into Leu or into Tyr; Ser into Thr; Thr into Ser; Trp into Tyr; Tyr into Trp; and/or Phe into Val, into IIe or into Leu. Hence, in one embodiment, a sequence having a given percentage sequence identity as given herein before is a sequence having one, two, three or more conservative amino acid substitutions as compared to the reference sequence.

On the other hand, variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property.

As recognized by those skilled in the art, (nucleic acids encoding) protein fragments, functional protein domains, and homologous proteins are also considered to be within the scope of tumor antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to a tumor-associated antigen. In addition to variants that are identical to the reference protein but are truncated, in some embodiments, an antigen may include 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

The present invention also provides mouse-specific nucleic acid molecules encoding for immunostimulatory proteins, co-stimulatory molecules, cytokines, and/or chemokines as set forth in Table 3.

Hence, in a particular embodiment, the mouse nucleic acid sequences described herein are at least and/or about 75%, 80%, 85%, 90%, 91%, 92%, 93%, 94%, 95%, 96%, 97%, 98%, 99% or 100% identical to the SEQ IDs recited in Table 3.

Within the context of the present invention, it is important to understand that the composition comprises at least one nucleic acid molecule encoding the immunostimulatory protein IL-21. In addition, the composition may comprise at least one or more nucleic acid sequences encoding for another immunostimatory protein such as a co-stimulatory molecule, a cytokine, and/or a chemokine. Therefore, the composition may comprise one or more nucleic acid sequences encoding for solely IL-21, or it may comprise two or more nucleic acid molecules in case an additional immunostimulatory protein is encoded. Alternatively, IL-21 and the at least one other immunostimulatory protein may be encoded by one single nucleic acid molecule.

Where the composition comprises nucleic acids encoding two or more proteins as defined herein, these may be encoded from a single nucleic acid molecule or from two or more nucleic acid molecules.

In a particular embodiment, the two or more nucleic acid molecules, in particular mRNA or DNA molecules, encoding the immunostimulatory proteins are thus separate nucleic acid molecules. In another embodiment, said mRNA or DNA molecules are part of one single nucleic acid molecule, wherein the single nucleic acid molecule is capable of expressing the two or more immunostimulatory proteins simultaneously. This single mRNA or DNA molecule is preferably capable of expressing the two or more proteins independently. In a preferred embodiment, the two or more mRNA or DNA molecules encoding the immunostimulatory proteins are linked in the single mRNA or DNA molecule by an internal ribosomal entry site (IRES) enabling separate translation of each of the two or more mRNA sequences into an amino acid sequence. Alternatively, a self-cleaving 2a peptide encoding sequence is incorporated between the coding sequences of the different immunostimulatory factors. This way, two or more factors can be encoded by one single mRNA or DNA molecule.

The invention thus further provides for an mRNA molecule encoding two or more immunostimulatory factors, wherein the two or more immunostimulatory factors are either translated separately from the single mRNA molecule through the use of an IRES between the two or more coding sequences. Alternatively, the invention provides an mRNA molecule encoding two or more immunostimulatory factors separated by a selfcleaving 2a peptide-encoding sequence, enabling the cleavage of the two protein sequences after translation.

In certain embodiments, the nucleic acid molecule in the composition is selected from the group comprising RNA, DNA, preferably RNA, more preferably mRNA.

A “nucleic acid” in the context of the invention is a deoxyribonucleic acid (DNA) or preferably a ribonucleic acid (RNA), more preferably mRNA but may also comprise cDNA, recombinantly produced and chemically synthesized molecules. A nucleic acid may according to the invention be in the form of a molecule which is single stranded or double stranded and linear or closed covalently to form a circle. A nucleic acid can be employed for introduction into, i.e. transfection of cells, for example, in the form of RNA which can be prepared by in vitro transcription from a DNA template. The RNA can moreover be modified before application by stabilizing sequences, capping, and/or polyadenylation.

In the context of the present invention, the term “RNA” relates to a molecule which comprises ribonucleotide residues and preferably being entirely or substantially composed of ribonucleotide residues.

“Ribonucleotide” relates to a nucleotide with a hydroxyl group at the 2′-position of a B-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs. Nucleic acids may be comprised in a vector. The term “vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available.

In a particular embodiment, the nucleic acid molecule of the composition comprises one or more of the following: a 5′CAP, a poly(A) tail and/or modified nucleoside(s); wherein said modified nucleoside is N1-methylpseudouridine.

In the context of the present invention, the term “five-prime cap (5CAP)” is to be understood as a specially altered nucleotide on the 5′end of some primary transcripts such as precursor messenger RNA that is synthesized during an mRNA capping process. In a particular embodiment, in vitro transcribed mRNA molecules might have a 5′ CAP-1, 5′ CAP-2, 5′ m6Am structure, or derivatives thereof. The eukaryotic 5′ cap consists of a 7-methylguanosine (m7G) connected by a triphosphate bridge to the first nucleotide, forming a structure known as ARCA cap analog (5′ CAP-0 analog). In the context of the present invention, the term “5′ CAP-1” (CleanCap) is meant to be a CAP-0 structure with an additional methyl group (2′ mono methylated) at the second carbon of the ribose sugar of the first cap-proximal nucleotide, such as represented herein below:

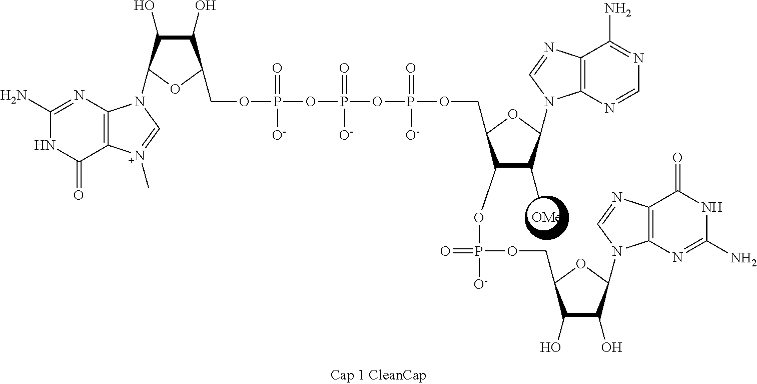

In the context of the present invention, the term “5′ CAP-2” is meant to be a CAP-1 structure with an additional methyl group (2′ dimethylated) at the second carbon of the ribose sugar of the second cap-proximal nucleotide. In the context of the present invention, the term “5′ m6Am” structure is meant to be a CAP-1 structure wherein the first nucleotide is an adenosine with a methyl group at the sixth nitrogen forming N6-methyladenosine (m6Am). As used herein, capping may involve a capping strategy during the mRNA manufacturing. In a specific embodiment, capping may refer to ‘co-transcriptional capping’ wherein a cap analog is incorporated during transcription. For example, CleanCap® technology is a proprietary, co-transcriptional 5′ capping solution that generates a natural Cap 1 structure. In another embodiment, capping may refer to ‘posttranscriptional capping’ wherein a cap analog is incorporated after the mRNA synthesis using an enzyme-based method.

The term “nucleosides” means nucleotides without a phosphate group. In a further embodiment, one or more of the mRNA molecules of the present invention may further comprise at least one modified nucleoside. In another particular embodiment, two, three, four, . . . or all of the used mRNA molecules of the present invention have at least one modified nucleoside.

In another particular embodiment of the present invention, said mRNA molecules further comprise at least one modified nucleoside, such as selected from the list comprising pseudouridine, 5-methoxy-uridine, 5-methyl-cytidine, 2-thio-uridine, and N6-methyladenosine.

In a particular embodiment of the present invention, said at least one modified nucleoside may be a pseudouridine, such as selected from the list comprising: 4-thio-pseudouridine, 2-thio-pseudouridine, 1-carboxymethyl-pseudouridine, 1-ethyl-pseudouridine; 1-propynyl-pseudouridine, 1-taurinomethyl-pseudouridine, N1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 4-methoxy-pseudouridine, and 4-methoxy-2-thio-pseudouridine. In a very specific embodiment, said at least one modified nucleoside is N1-methyl-pseudouridine.

Alternative nucleoside modifications which are suitable for use within the context of the invention, include: pyridin-4-one ribonucleoside, 5-aza-uridine, 2-thio-5-aza-uridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, -thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6, N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, I-methyl-6-thio-guaiguanosine, and N2, N2-dimethyl-6-thio-guanosine.

The mRNA molecules used in the present invention may contain one or more modified nucleotides, in particular embodiment, at least 10%, 20%, 30%, 40%, 50%, 60%, 70%, 80%, 90% or 100% of a particular type of nucleotides may be replaced by a modified one. It is also not excluded that different nucleotide modifications are included within the same mRNA molecule. In a very specific embodiment of the present invention, about 100% of uridines in said mRNA molecules is replaced by N1-methyl-pseudouridine.

As used herein, the term “poly(A) tail” is to be understood as a moiety comprising multiple adenosine monophosphates and is well known in the art. A poly(A) tail is generally produced during a step called polyadenylation that is one of the post-translation modifications which generally occur during the production of mature messenger RNAs; such poly(A) tail contribute to the stability and the half-life of said mRNAs, and can be of variable length. In particular, a poly(A) tail may be equal or longer than 10 adenosine nucleotides, which includes equal or longer than 20 adenosine nucleotides, which includes equal or longer than 100 adenosine nucleotides, and for example about between 90 and 120 adenosine nucleotides, such as about 90 or about 120 adenosine nucleotides.

The nucleic acid molecules used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection, lipid nanoparticles, etc.

The present invention also provides a composition as defined herein; wherein one or more of said mRNA molecules are encompassed in nanoparticles.

As used herein, the term “nanoparticle” refers to any particle having a diameter making the particle suitable for systemic, in particular intratumoral, intramuscular or intravenous administration, of, in particular, nucleic acids, typically having a diameter of less than 1000 nanometers (nm), preferably less than 500 nm, even more preferably less than 200 nm, such as for example between 50 and 200 nm; preferably between 80 and 160 nm.

In a specific embodiment of the present invention, the nanoparticles are selected from the list comprising: lipid nanoparticles and polymeric nanoparticles.

The invention further provides a lipid nanoparticle comprising the composition as described by the invention.

In the context of the present invention, by means of the term “lipid nanoparticle”, or LNP, reference is made to a nanosized particle composed of one or more lipids, e.g. a combination of different lipids. Possible lipids used in the LNP can be for example, but not limited to at least one phospholipids, at least one polymer-modified lipid such a PEG lipid or polysarcosine lipid, at least one cationic or ionisable lipid, at least one sterol.

In the context of the present invention the term “ionisable” (or alternatively cationic) in the context of a compound or lipid means the presence of any uncharged group in said compound or lipid which is capable of dissociating by yielding an ion (usually an H+ion) and thus itself becoming positively charged. Alternatively, any uncharged group in said compound or lipid may yield an electron and thus becoming negatively charged. The pH-sensitivity of ionizable lipids is beneficial for mRNA delivery in vivo, because neutral lipids have less interactions with the anionic membranes of blood cells and, thus, improve the biocompatibility of lipid nanoparticles. As used herein, any type of ionizable lipid can suitably be used. For example, suitable ionizable lipids are ionizable amino lipids which comprise 2 identical or different tails linked via an S—S bond.

In the context of the present invention, the term “PEG lipid” or alternatively “PEGylated lipid” is meant to be any suitable lipid modified with a PEG (polyethylene glycol) group. In the context of the present invention, the term “phospholipid” is meant to be a lipid molecule consisting of two hydrophobic fatty acid “tails” and a hydrophilic “head” consisting of a phosphate groups. The two components are most often joined together by a glycerol molecule, hence, in the phospholipid of the present invention is preferably a glycerol-phospholipid.

In the context of the present invention, the term “sterol”, also known as steroid alcohol, is a subgroup of steroids that occur naturally in plants, animal and fungi, or can be produced by some bacteria. In the context of the present invention, any suitable sterol may be used, such as for example cholesterol.

In a specific embodiment of the present invention one or more of the following applies:
said LNP comprises about and between 10 mol % and 70 mol % of said ionizable lipid;
said LNP comprises about and between 1 mol % and 40 mol % of said phospholipid;
said LNP comprises about and between 0.5 mol % and 10 mol % of said PEG lipid;
   balanced by the amount of said sterol.

In a preferred embodiment of the present invention one or more of the following applies:
said LNP comprises about and between 35 mol % and 65 mol % of said ionizable lipid;
said LNP comprises about and between 5 mol % and 25 mol % of said phospholipid;
said LNP comprises about and between 0.5 mol % and 3.0 mol % of said PEG lipid;
   balanced by the amount of said sterol.

In another preferred embodiment, the LNP of the present invention comprises 50 mol % of ionizable lipid, 10 mol % of phospholipid, 1.5 mol % of PEG lipid and 38.5 mol % of sterol.

In some embodiments, the mixture of lipids forms lipid nanoparticles. In some embodiments, the composition of the present invention is formulated in the lipid nanoparticles. In some embodiments, the lipid nanoparticles are formed first as empty lipid nanoparticles and combined with the composition immediately prior to (e.g., within a couple of minutes to an hour of) administration, in particular a vaccine administration.

To avoid any misunderstanding, the LNP's of the present invention may comprise a composition, or they may comprise a plurality of compositions, such as a combination of one or more compositions comprising nucleic acids encoding immune modulating proteins; and/or one or more compositions comprising nucleic acids encoding a co-stimulatory molecule, a cytokine, and/or a chemokine.

In a very specific embodiment, the LNP's of the present invention may comprise a composition comprising nucleic acids encoding immunomodulatory molecules and one or more nucleic acid molecules encoding peptides derived from tumor or cancer cells. For example, the LNP's of the present invention may comprise a composition comprising nucleic acids encoding peptides derived from a tumor; in combination with one or more nucleic acids encoding the immunostimulatory protein IL-21, optionally in combination with one or more other nucleic acid molecules encoding a co-stimulatory molecule, a cytokine, and/or a chemokine.

Furthermore, it should be understood that the LNP's of the present invention may comprise said composition, said nucleic acid molecules or said pharmaceutical formulation according to the present invention.

In some embodiments, two or more different nucleic acid molecules (e.g., mRNA) encoding immunostimulatory proteins and/or tumor antigens may be formulated in the same lipid nanoparticle. In other embodiments, two or more different nucleic acid molecules encoding immunostimulatory proteins or tumor antigens may be formulated in separate lipid nanoparticles (each nucleic acid formulated in a single lipid nanoparticle). The lipid nanoparticles may then be combined and administered as a single composition (e.g., comprising multiple nucleic acid encoding multiple immunostimulatory proteins or tumor antigens) or may be administered separately.

The composition, nucleic acid molecules, or pharmaceutical formulation defined herein can be formulated in lipid nanoparticles (LNPs) that encapsulate the constructs to protect them from degradation and promote cellular uptake.

The invention further provides a pharmaceutical formulation comprising the composition or the lipid nanoparticle and at least one pharmaceutically acceptable carrier or excipient.

In particular, as defined herein, the term “pharmaceutical formulation” is in particular used in the context of said LNP comprising the composition of the invention in combination with at least one pharmaceutically acceptable carrier or excipient. On the other hand, the term “pharmaceutical formulation” may also refer to a combination of the composition of the invention and at least one pharmaceutically acceptable carrier or excipient (i.e. without encapsulation within the LNP). This combination can for example be formulated in a LNP and is in particularly intended for prolonging nucleic acid stability and/or improve delivery.

As used herein, a “formulation”, refers to any mixture of two or more products or compounds (e.g. agents, modulators, regulators, etc.). It can be a solution, a suspension, liquid, or aqueous formulations or any combination thereof.

In the context of the present invention, by means of the term “pharmaceutical formulation” reference is made to a formulation having pharmaceutical properties. In other words, reference is made to a formulation providing for a pharmacological and/or physiological effect. Pharmaceutical formulations can comprise one or more pharmaceutically acceptable agents such as excipients, carriers, diluents.

In some embodiments, the pharmaceutically acceptable agents include, but are not limited to, biocompatible vehicles, adjuvants, additives, and diluents to achieve a composition usable as a dosage form.

As used herein and unless otherwise specified, the term “excipient” is to be understood as any substance formulated alongside the active compound included for the purpose of long-term stabilization such as prevention of denaturation or aggregation over the expected shelf life, bulking up liquid or solid formulations that contain potent active compound in small amounts (thus often referred to as “bulking agents”, “fillers”, or “diluents”), or to confer an enhancement on the active compound in the final dosage form, such as facilitating absorption, reducing viscosity, or enhancing solubility.

Specific formulations of the present invention may for example comprise:

| | | |
|---|---|---|
| Buffer | Phosphate (potassium dihydrogen | Tris |
| | phosphate, disodium | (tromethamine) |
| | hydrogen phosphate dihydrate) | |
| Other | Potassium chloride, | Sodium |
| excipients | sodium chloride | acetate |
| | Sucrose | Sucrose |
| | Water for injection | Water for injection |

Pharmaceutical formulations are particularly suitable as a vaccine

In the context of the present invention, the term “vaccine” as used herein is meant to be any preparation intended to provide adaptive immunity (antibodies and/or T cell responses) against a disease. To that end, the term “vaccine” as meant herein comprises at least one composition or at least one lipid nanoparticle, or at least one pharmaceutical formulation, to which an adaptive immune response is mounted. Within the context of the present invention, the term vaccine can be used interchangeably with the term pharmaceutical formulation.

In some embodiments, said vaccine may comprise naked nucleic acids which are suspended in a suitable injection buffer, such as a Ringer Lactate buffer.

The vaccines of the present invention may be used prophylactic (such as prior to the manifestation of symptoms), or therapeutic (example, to actively treat or reduce the symptoms of an ongoing disease such as tumor growth). The administration of vaccines is called vaccination.

In a further aspect, the present invention provides a composition, a lipid nanoparticle or a pharmaceutical formulation for use in parenteral administration; more in particular for use in vaccine administration routs known in the art such as intravenous, intratumoral, intradermal, intraperitoneal, intramuscular or intranodal administration, preferably intratumoral administration.

The vaccine of the present invention is in particular intended for intratumural administration, i.e. the infusion of liquid substance directly into the tumor. Fluids administered into the tumor are rapidly absorbed into the circulation, i.e. systematically.

The present invention also provides a vaccine being administered intravenously, i.e. the infusion of liquid substance directly into a vein. The intravenous route is the fastest way to deliver fluids and medications throughout the body but leads to a widespread systemic exposure.

Within the context of the present invention, the vaccine may be administered prophylactically (=preventive) or therapeutically as part of an active vaccination scheme to individuals that are early in the disease onset, after onset of symptoms, or after detecting expression or one or more tumor antigens.

In some embodiments, the vaccine may be administered as a monotherapy or as a combination therapy. As described in the example part, the term “monotherapy” is to be understood as a vaccine administration comprising a nucleic acid encoding for one single immunostimulatory protein, e. g. IL-21. In contract, the term “combination therapy” is meant to be as a vaccine administration comprising a nucleic acid encoding for two or more immunostimulatory protein, e. g. IL-21 in combination with IL-7.

Alternatively, combination therapy may also be used in the context of the composition of the invention encoding IL-21 either or not in combination with other immunostimulatory molecules or other h standard of care tumour treatments such as chemotherapy or radiotherapy

In another embodiment, the therapeutic of the invention comprising a nucleic acid encoding for one or more immunomodulatory proteins may also be administered in combination with antigen-specific tumour vaccines resulting in a “combined treatment” effect . . .

In some embodiments, the vaccine (as a monotherapy or combination therapy) may be administered as a single dose, two doses, three doses, four doses, preferably three doses, or repeated as long as the subject is in need thereof.

In some embodiments, the time of administration in a monotherapy or combination therapy may be, but is not limited to 1 week, 2 weeks, 3 weeks, 4 weeks, 2 months, 3 months, 6 months, 1 year and may be repeated every year, as long as the subject is in need thereof.

In another embodiment, the time of administration between the injections in a combined treatment (i.e. mono/combinational therapy with another anti-tumor vaccine) may be, but is not limited to 1 minute to 30 minutes, 30 minutes to 1 hours, 3 hours, 6 hours, 12 hours, 1 day, 1 week, 2 weeks, 3 weeks, 4 weeks, 2 months, 3 months, 6 months, 1 year.

For each of said administrations, the dosing may also be varied, such as a higher dose at the beginning of the treatment, and a lower dose towards the end of the treatment.

In a particular embodiment, the present invention also provides a composition, a lipid nanoparticle, a pharmaceutical formulation or a vaccine for use in human or veterinary medicine.

The use of a composition, a lipid nanoparticle, a pharmaceutical formulation or a vaccine as defined herein in human or veterinary medicine is also intended. Finally, the invention provides a method for the prophylaxis and/or treatment of human and veterinary disorders, by administering a composition, a lipid nanoparticle, a pharmaceutical formulation or a vaccine as defined herein to a subject in need thereof.

In a more preferred embodiment, a composition, a lipid nanoparticle or a pharmaceutical formulation as defined herein is provided for use in the prevention and/or treatment of cell proliferative disorders.

In the context of the present application, the terms “treatment”, “treating”, “treat” and the like refer to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” covers any treatment of a disease in a mammal, in particular a human, and includes: (a) preventing the disease or symptom from occurring in a subject which may be predisposed to the disease or symptoms but has not yet been diagnosed as having it; (b) inhibiting the disease symptoms, i.e. arresting its development; or (c) relieving the disease symptom, i.e. causing regression of the disease or symptom.

In yet another embodiment, a composition, a lipid nanoparticle or a pharmaceutical formulation as defined herein is provided for use in eliciting an immune response towards a tumor and/or cancer in a subject.

The term “immune response” used throughout the description is not intended to be limited to the types of immune responses that may have been exemplified herein. The term therefore encompasses all tumor antigens or cancer antigens to which vaccination would be beneficial to the subject.

The vaccine of the invention may be used for inducing an immune response, in particular an immune response against a disease-associated antigen or cells expressing a disease-associated antigen, such as an immune response against tumor-associated antigens. Preferably said immune response is a T cell response. In one embodiment, the disease-associated antigen is a tumor antigen. The antigen encoded by the nucleic acids of the present invention described herein preferably is a disease-associated antigen or elicits an immune response against a disease-associated antigen or cells expressing a disease-associated antigen.

In a preferred embodiment, the present invention provides a composition that is used for direct in vivo application.

Through direct in vivo delivery, a strong activation stimulus for DCs is provided, resulting in the induction of a T cell attracting and stimulatory environment. Moreover, when an antigen mRNA is co-delivered together with the composition of the present invention, this results in the recruitment of antigen-specific CD4+ and CD8+ T cells as well as CTLs against various tumor antigens. In particular, in vivo delivery of said composition initiates maturation of DCs after the uptake and translation of the mRNA as such still allowing strong antigen expression by these DCs. Simultaneous delivery of said composition and antigen mRNA significantly enhances the induction of antigen-specific T cells compared to direct in vivo delivery of antigen mRNA alone. These observations point toward the value of co-delivering the mRNA composition for the induction of strong antigen-specific immune responses against cancer.

In a preferred embodiment, a composition, a lipid nanoparticle or a pharmaceutical formulation as defined herein is provided for use in combination with standard cancer therapies including radiotherapy and chemotherapy.

The invention further provides for methods of treating a patient in need thereof with a composition, a lipid nanoparticle or a pharmaceutical formulation of the invention or with the vaccine of the invention.

The invention further provides a composition, lipid nanoparticle or pharmaceutical formulation as defined herein for treating cancer. In case of active immunotherapy for cancer, the treatment with the composition, the lipid nanoparticle or the pharmaceutical formulation of the invention can be preceded by, combined with or followed by any non-specific treatment of immunomodulation in order to improve the activity of the invention itself or to exploit any synergy between the different treatment modalities (e.g. by improving the immune response to the invention through non-specific stimulation of the patient's immune system with cytokines (e.g. interleukin-2 or Interferon alfa-2b) or TLR-ligands; or e.g. by combination of the invention with a co-stimulatory signal modifying drug such as ipilimumab or tremelimumab); or any other form of immunotherapy. The invention also provides for complex treatment regimens in which the invention itself and a defined number of other immunomodulatory treatments are used to result in a more active treatment plan (e.g. the sequential use of the invention with modality 1 (e.g. a cytokine) followed by the use of the invention for in vivo or ex vivo expansion of vaccinal immune cells followed by an adoptive cellular transfer of these cells followed by a combination treatment of the invention with an additional modality (e.g. a costimulatory receptor signal modifier) or any possible combination of concomitant and/or sequential use of the invention and additional immunomodulatory treatments.

In a preferred embodiment, the invention lies in the simultaneous expression of IL-21 with a target-specific antigen, thereby leading to increased immunostimulatory effects of the DCs in the tumor environment. In a further preferred embodiment, the specific combination of IL-21, 4-1BBL and IL-7 is used to improve the immunostimulatory effects of the DCs. In another preferred embodiment, the specific combination of IL-21, 4-1BBL and IL-15sushi is used to improve the immunostimulatory effects of the DCs. In all of these embodiments, any of the following markers could be introduced simultaneously: a cytokine, and/or a chemokine,

TABLE 1
Human amino acid sequences of encoded immunostimulatory proteins.

| SEQ | Peptide or | NCBI | |
|---|---|---|---|
| ID | fragment | number | Amino acid sequence |
| 1 | hulL-21 iso | NP_068575.1 | MRSSPGNMERIVICLMVIFLGTLVHKSSSQGQDRHMIRMRQLIDIVDQLKNYVNDLVP |
| | 1 | | EFLPAPEDVETNCEWSAFSCFQKAQLKSANTGNNERIINVSIKKLKRKPPSTNAGRRQ |
| | | | KHRLTCPSCDSYEKKPPKEFLERFKSLLQKMIHQHLSSRTHGSEDS |
| 2 | huIL-21 iso | NP_001193935 | MRSSPGNMERIVICLMVIFLGTLVHKSSSQGQDRHMIRMRQLIDIVDQLKNYVNDLVP |
| | 2 | | EFLPAPEDVETNCEWSAFSCFQKAQLKSANTGNNERIINVSIKKLKRKPPSTNAGRRQ |
| | | | KHRLTCPSCDSYEKKPPKEFLERFKSLLQKVSTLSFI |
| 3 | hulL-7 iso | NP_000871.1 | MFHVSFRYIFGLPPLILVLLPVASSDCDIEGKDGKQYESVLMVSIDQLLDSMKEIGSNC |
| | 1 | | LNNEFNFFKRHICDANKEGMFLFRAARKLRQFLKMNSTGDFDLHLLKVSEGTTILLNC |
| | | | TGQVKGRKPAALGEAQPTKSLEENKSLKEQKKLNDLCFLKRLLQEIKTCWNKILMGTK |
| | | | EH |
| 4 | hull-7 iso | NP_ | MFHVSFRYIFGLPPLILVLLPVASSDCDIEGKDGKQYESVLMVSIDQLLDSMKEIGSNC |
| | 2 | 001186815.1 | LNNEFNFFKRHICDANKVKGRKPAALGEAQPTKSLEENKSLKEQKKLNDLCFLKRLLQ |
| | | | EIKTCWNKILMGTKEH |
| 5 | hu4-1BB-L | NP_003802.1 | MEYASDASLDPEAPWPPAPRARACRVLPWALVAGLLLLLLLAAACAVFLACPWAVSG |
| | (tnfsf9) | | ARASPGSAASPRLREGPELSPDDPAGLLDLRQGMFAQLVAQNVLLIDGPLSWYSDP |
| | | | GLAGVSLTGGLSYKEDTKELVVAKAGVYYVFFQLELRRVVAGEGSGSVSLALHLQPL |
| | | | RSAAGAAALALTVDLPPASSEARNSAFGFQGRLLHLSAGQRLGVHLHTEARARHAW |
| | | | QLTQGATVLGLFRVTPEIPAGLPSPRSE |
| 6 | huIL-18 | NP_001553.1 | MAAEPVEDNCINFVAMKFIDNTLYFIAEDDENLESDYFGKLESKLSVIRNLNDQVLFID |
| | WT | | QGNRPLFEDMTDSDCRDNAPRTIFIISMYKDSQPRGMAVTISVKCEKISTLSCENKIISF |
| | | | KEMNPPDNIKDTKSDIIFFQRSVPGHDNKMQFESSSYEGYFLACEKERDLFKLILKKED |
| | | | ELGDRSIMFTVQNED |
| 7 | huCCL5 | NP_002976 | MKVSAAALAVILIATALCAPASASPYSSDTTPCCFAYIARPLPRAHIKEYFYTSGKCSNP |
| | | | AVVFVTRKNRQVCANPEKKWVREYINSLEMS |
| 8 | huCCL19 | NP_006265.1 | MALLLALSLLVLWTSPAPTLSGTNDAEDCCLSVTQKPIPGYIVRNFHYLLIKDGCRVPA |
| | | | VVFTTLRGRQLCAPPDQPWVERIIQRLQRTSAKMKRRSS |
| 9 | huCXCL9 | NP_002407.1 | MKKSGVLFLLGIILLVLIGVQGTPVVRKGRCSCISTNQGTIHLQSLKDLKQFAPSPSCEK |
| | | | IEIIATLKNGVQTCLNPDSADVKELIKKWEKQVSQKKKQKNGKKHQKKKVLKVRKSQR |
| | | | SRQKKTT |
| 10 | huXCL1 | NP_002986.1 | MRLLILALLGICSLTAYIVEGVGSEVSDKRTCVSLTTQRLPVSRIKTYTITEGSLRAVIFIT |
| | | | KRGLKVCADPQATWVRDVVRSMDRKSNTRNNMIQTKPTGTQQSTNTAVTLTG |
| 11 | huXCL1- | Not | MRLLILALLGICSLTAYIVEGVGSEVSDKRTCVSLTTQRLPCSRIKTYTITEGSLRAVIFIT |
| | V21C/V59C | available | KRGLKVCADPQATWVRDCVRSMDRKSNTRNNMIQTKPTGTQQSTNTAVTLTG |
| 12 | huIL-29 | NP_742152.1 | MAAAWTVVLVTLVLGLAVAGPVPTSKPTTTGKGCHIGRFKSLSPQELASFKKARDALE |
| | (Interferon | | ESLKLKNWSCSSPVFPGNWDLRLLQVRERPVALEAELALTLKVLEAAAGPALEDVLD |
| | lambda 1) | | QPLHTLHHILSQLQACIQPQPTAGPRPRGRLHHWLHRLQEAPKKESAGCLEASVTFN |
| | | | LFRLLTRDLKYVADGNLCLRTSTHPEST |
| 13 | huIL-28A | NP_742150.1 | MKLDMTGDCTPVLVLMAAVLTVTGAVPVARLHGALPDARGCHIAQFKSLSPQELQAF |
| | (Interferon | | KRAKDALEESLLLKDCRCHSRLFPRTWDLRQLQVRERPMALEAELALTLKVLEATADT |
| | lambda 2) | | DPALVDVLDQPLHTLHHILSQFRACIQPQPTAGPRTRGRLHHWLYRLQEAPKKESPG |
| | | | CLEASVTFNLFRLLTRDLNCVASGDLCV |
| 14 | huIL-28B | NP_742151.2 | MTGDCMPVLVLMAAVLTVTGAVPVARLRGALPDARGCHIAQFKSLSPQELQAFKRAK |
| | (Interferon | | DALEESLLLKDCKCRSRLFPRTWDLRQLQVRERPVALEAELALTLKVLEATADTDPAL |
| | lambda 3) | | GDVLDQPLHTLHHILSQLRACIQPQPTAGPRTRGRLHHWLHRLQEAPKKESPGCLEA |
| | | | SVTFNLFRLLTRDLNCVASGDLCV |

TABLE 2
Mouse amino acid sequences of encoded immunostimulatory proteins.

| SEQ | Peptide or | NCBI | |
|---|---|---|---|
| ID | fragment | number | Amino acid sequence |
| 15 | muIL-21 iso1 | NP_001277970.1 | MERTLVCLVVIFLGTVAHKSSPQGPDRLLIRLRHLIDIVEQLKIYENDLDPELLS |
| | | | APQDVKGHCEHAAFACFQKAKLKPSNPGNNKTFIIDLVAQLRRRLPARRGGK |
| | | | KQKHIAKCPSCDSYEKRTPKEFLERLKWLLQKVCTLNAFLSLPCCVRVPPVP |
| | | | SDS |
| 16 | muIL-21 iso2 | NP_068554.1 | MERTLVCLVVIFLGTVAHKSSPQGPDRLLIRLRHLIDIVEQLKIYENDLDPELLS |
| | | | APQDVKGHCEHAAFACFQKAKLKPSNPGNNKTFIIDLVAQLRRRLPARRGGK |
| | | | KQKHIAKCPSCDSYEKRTPKEFLERLKWLLQKMIHQHLS |
| 17 | muIL-7 | NP_032397.1 | MFHVSFRYIFGIPPLILVLLPVTSSECHIKDKEGKAYESVLMISIDELDKMTGTD |
| | | | SNCPNNEPNFFRKHVCDDTKEAAFLNRAARKLKQFLKMNISEEFNVHLLTVS |
| | | | QGTQTLVNCTSKEEKNVKEQKKNDACFLKRLLREIKTCWNKILKGSI |
| 18 | mu4-1BB-L | NP_033430.1 | MDQHTLDVEDTADARHPAGTSCPSDAALLRDTGLLADAALLSDTVRPTNAAL |
| | (tnfsf9) | | PTDAAYPAVNVRDREAAWPPALNFCSRHPKLYGLVALVLLLLIAACVPIFTRT |
| | | | EPRPALTITTSPNLGTRENNADQVTPVSHIGCPNTTQQGSPVFAKLLAKNQA |
| | | | SLCNTTLNWHSQDGAGSSYLSQGLRYEEDKKELVVDSPGLYYVFLELKLSPT |
| | | | FTNTGHKVQGWVSLVLQAKPQVDDFDNLALTVELFPCSMENKLVDRSWSQL |
| | | | LLLKAGHRLSVGLRAYLHGAQDAYRDWELSYPNTTSFGLFLVKPDNPWE |
| 19 | muIgGk signal | Not available | METDTLLLWVLLLWVPGSTGDTTCPPPVSIEHADIRVKNYSVNSRERYVCNS |
| | peptide-mu | | GFKRKAGTSTLIECVINKNTNVAHWTTPSLKCIRDPSLAGGSGGSGGSGGSG |
| | IL-15Ra sushi | | GSGGSGGNWIDVRYDLEKIESLIQSIHIDTTLYTDSDFHPSCKVTAMNCFLLEL |
| | (aa34-103)- | | QVILHEYSNMTLNETVRNVLYLANSTLSSNKNVAESGCKECEELEEKTFTEFL |
| | GSlinker-IL-15 | | QSFIRIVQMFINTS |
| | (aa49-162) | | |
| 20 | muIL-18 WT | NP_032386.1 | MAAMSEDSCVNFKEMMFIDNTLYFIPEENGDLESDNFGRLHCTTAVIRNINDQ |
| | | | VLFVDKRQPVFEDMTDIDQSASEPQTRLIIYMYKDSEVRGLAVTLSVKDSKMS |
| | | | TLSCKNKIISFEEMDPPENIDDIQSDLIFFQKRVPGHNKMEFESSLYEGHFLAC |
| | | | QKEDDAFKLILKKKDENGDKSVMFTLTNLHQS |
| 21 | gausia signal | Not available | MGVKVLFALICIAVAEANFGRLHCTTAVIRNINDQVLFVDKRQPVFEDMTDIDQ |
| | peptide-muIL- | | SASEPQTRLIIYMYKDSEVRGLAVTLSVKDSKMSTLSCKNKIISFEEMDPPENI |
| | 18 WT | | DDIQSDLIFFQKRVPGHNKMEFESSLYEGHFLACQKEDDAFKLILKKKDENGD |
| | | | KSVMFTLTNLHQS |
| 22 | muIL-18 | Not | MAAMSEDSCVNFKEMMFIDNTLYFIPEENGDLESDHFGRLHCTTAVIRNINDQ |
| | mutant CS2 | available/from | VLFVDKRQPVFEDMTDIDQSASEPQTRLIIYAYGDSRARGKAVTLSVKDSKM |
| | (Decoy | literature | STLSCKNKIISFEEMDPPENIDDIQSDLIFFQKRVPGHNKMEFESSLYEGHFLA |
| | Receptor) | | CQKEDDAFKLILKKKDENGDKSVMFTLTNLHQS |
| 23 | gausia signal | Not available | MGVKVLFALICIAVAEAHFGRLHCTTAVIRNINDQVLFVDKRQPVFEDMTDIDQ |
| | peptide-muIL- | | SASEPQTRLIIYAYGDSRARGKAVTLSVKDSKMSTLSCKNKIISFEEMDPPENI |
| | 18 mutant | | DDIQSDLIFFQKRVPGHNKMEFESSLYEGHFLACQKEDDAFKLILKKKDENGD |
| | CS2 (Decoy | | KSVMFTLTNLHQS |
| | Receptor) | | |
| 24 | muCCL5 | NP_038681.2 | MKISAAALTIILTAAALCTPAPASPYGSDTTPCCFAYLSLALPRAHVKEYFYTS |
| | | | SKCSNLAVVFVTRRNRQVCANPEKKWVQEYINYLEMS |
| 25 | muCCL19 | NP_036018.1 | MAPRVTPLLAFSLLVLWTFPAPTLGGANDAEDCCLSVTQRPIPGNIVKAFRYL |
| | | | LNEDGCRVPAVVFTTLRGYQLCAPPDQPWVDRIIRRLKKSSAKNKGNSTRRS |
| | | | PVS |
| 26 | muCXCL9 | NP_032625.2 | MKSAVLFLLGIIFLEQCGVRGTLVIRNARCSCISTSRGTIHYKSLKDLKQFAPS |
| | | | PNCNKTEIIATLKNGDQTCLDPDSANVKKLMKEWEKKISQKKKQKRGKKHQK |
| | | | NMKNRKPKTPQSRRRSRKTT |
| 27 | muXCL1 | NP_032536.1 | MRLLLLTFLGVCCLTPWVVEGVGTEVLEESSCVNLQTQRLPVQKIKTYIIWEG |
| | | | AMRAVIFVTKRGLKICADPEAKWVKAAIKTVDGRASTRKNMAETVPTGAQRS |
| | | | TSTAITLTG |
| 28 | muXCL1- | Not available | MRLLLLTFLGVCCLTPWVVEGVGTEVLEESSCVNLQTQRLPCQKIKTYIIWEG |
| | V21C/A59C | | AMRAVIFVTKRGLKICADPEAKWVKACIKTVDGRASTRKNMAETVPTGAQRS |
| | | | TSTAITLTG |
| 29 | muIL-28A | NP_001019844.2 | MLLLLLPLLLAAVLTRTQADPVPRATRLPVEAKDCHIAQFKSLSPKELQAFKKA |
| | (Interferon | | KDAIEKRLLEKDLRCSSHLFPRAWDLKQLQVQERPKALQAEVALTLKVWENM |
| | lambda 2) | | TDSALATILGQPLHTLSHIHSQLQTCTQLQATAEPRSPSRRLSRWLHRLQEA |
| | | | QSKETPGCLEASVTSNLFRLLTRDLKCVANGDQCV |
| 30 | muIL-28B | NP_796370.1 | MLLLLLPLLLAAVLTRTQADPVPRATRLPVEAKDCHIAQFKSLSPKELQAFKKA |
| | (Interferon | | KGAIEKRLLEKDMRCSSHLISRAWDLKQLQVQERPKALQAEVALTLKVWENI |
| | lambda 3) | | NDSALTTILGQPLHTLSHIHSQLQTCTQLQATAEPKPPSRRLSRWLHRLQEA |
| | | | QSKETPGCLEDSVTSNLFQLLLRDLKCVASGDQCV |

TABLE 3
Mouse nucleic acid sequences of encoded immunostimulatory proteins.

| | Peptide | | |
|---|---|---|---|
| | or | NCBI | |
| SEQ ID | fragment | number | Nucleotide sequences (DNA) |
| 31 | muIL-21 | NM_001291041.1 | ATGGAGAGGACCCTTGTCTGTCTGGTAGTCATCTTCTTGGGGACAGTGGCCC |
| | iso1 | | ATAAATCAAGCCCCCAAGGGCCAGATCGCCTCCTGATTAGACTTCGTCACCTT |
| | | | ATTGACATTGTTGAACAGCTGAAAATCTATGAAAATGACTTGGATCCTGAACTT |
| | | | CTATCAGCTCCACAAGATGTAAAGGGGCACTGTGAGCATGCAGCTTTTGCCT |
| | | | GTTTTCAGAAGGCCAAACTCAAGCCATCAAACCCTGGAAACAATAAGACATTC |
| | | | ATCATTGACCTCGTGGCCCAGCTCAGGAGGAGGCTGCCTGCCAGGAGGGGA |
| | | | GGAAAGAAACAGAAGCACATAGCTAAATGCCCTTCCTGTGATTCGTATGAGAA |
| | | | AAGGACACCCAAAGAATTCCTAGAAAGACTAAAATGGCTCCTTCAAAAGGTAT |
| | | | GCACCTTAAATGCATTTCTTTCACTTCCATGTTGTGTCCGGGTACCTCCTGTG |
| | | | CCCAGTGACTCATAG |
| 32 | muIL-21 | NM_021782.3 | ATGGAGAGGACCCTTGTCTGTCTGGTAGTCATCTTCTTGGGGACAGTGGCCC |
| | iso2 | | ATAAATCAAGCCCCCAAGGGCCAGATCGCCTCCTGATTAGACTTCGTCACCTT |
| | | | ATTGACATTGTTGAACAGCTGAAAATCTATGAAAATGACTTGGATCCTGAACTT |
| | | | CTATCAGCTCCACAAGATGTAAAGGGGCACTGTGAGCATGCAGCTTTTGCCT |
| | | | GTTTTCAGAAGGCCAAACTCAAGCCATCAAACCCTGGAAACAATAAGACATTC |
| | | | ATCATTGACCTCGTGGCCCAGCTCAGGAGGAGGCTGCCTGCCAGGAGGGGA |
| | | | GGAAAGAAACAGAAGCACATAGCTAAATGCCCTTCCTGTGATTCGTATGAGAA |
| | | | AAGGACACCCAAAGAATTCCTAGAAAGACTAAAATGGCTCCTTCAAAAGATGA |
| | | | TTCATCAGCATCTCTCCTAG |
| 33 | muIL-7 | NM_008371.5 | ATGTTCCATGTTTCTTTTAGATATATCTTTGGAATTCCTCCACTGATCCTTGTTC |
| | | | TGCTGCCTGTCACATCATCTGAGTGCCACATTAAAGACAAAGAAGGTAAAGCA |
| | | | TATGAGAGTGTACTGATGATCAGCATCGATGAATTGGACAAAATGACAGGAAC |
| | | | TGATAGTAATTGCCCGAATAATGAACCAAACTTTTTTAGAAAACATGTATGTGA |
| | | | TGATACAAAGGAAGCTGCTTTTCTAAATCGTGCTGCTCGCAAGTTGAAGCAAT |
| | | | TTCTTAAAATGAATATCAGTGAAGAATTCAATGTCCACTTACTAACAGTATCAC |
| | | | AAGGCACACAAACACTGGTGAACTGCACAAGTAAGGAAGAAAAAAACGTAAA |
| | | | GGAACAGAAAAAGAATGATGCATGTTTCCTAAAGAGACTACTGAGAGAAATAA |
| | | | AAACTTGTTGGAATAAAATTTTGAAGGGCAGTATATAA |
| 34 | mu4-1BB-L | NM_009404.3 | ATGGACCAGCACACACTTGATGTGGAGGATACCGCGGATGCCAGACATCCAG |
| | (tnfsf9) | | CAGGTACTTCGTGCCCCTCGGATGCGGCGCTCCTCAGAGATACCGGGCTCC |
| | | | TCGCGGACGCTGCGCTCCTCTCAGATACTGTGCGCCCCACAAATGCCGCGCT |
| | | | CCCCACGGATGCTGCCTACCCTGCGGTTAATGTTCGGGATCGCGAGGCCGC |
| | | | GTGGCCGCCTGCACTGAACTTCTGTTCCCGCCACCCAAAGCTCTATGGCCTA |
| | | | GTCGCTTTGGTTTTGCTGCTTCTGATCGCCGCCTGTGTTCCTATCTTCACCCG |
| | | | CACCGAGCCTCGGCCAGCGCTCACAATCACCACCTCGCCCAACCTGGGTAC |
| | | | CCGAGAGAATAATGCAGACCAGGTCACCCCTGTTTCCCACATTGGCTGCCCC |
| | | | AACACTACACAACAGGGCTCTCCTGTGTTCGCCAAGCTACTGGCTAAAAACCA |
| | | | AGCATCGTTGTGCAATACAACTCTGAACTGGCACAGCCAAGATGGAGCTGGG |
| | | | AGCTCATACCTATCTCAAGGTCTGAGGTACGAAGAAGACAAAAAGGAGTTGG |
| | | | TGGTAGACAGTCCCGGGCTCTACTACGTATTTTTGGAACTGAAGCTCAGTCCA |
| | | | ACATTCACAAACACAGGCCACAAGGTGCAGGGCTGGGTCTCTCTTGTTTTGC |
| | | | AAGCAAAGCCTCAGGTAGATGACTTTGACAACTTGGCCCTGACAGTGGAACT |
| | | | GTTCCCTTGCTCCATGGAGAACAAGTTAGTGGACCGTTCCTGGAGTCAACTG |
| | | | TTGCTCCTGAAGGCTGGCCACCGCCTCAGTGTGGGTCTGAGGGCTTATCTGC |
| | | | ATGGAGCCCAGGATGCATACAGAGACTGGGAGCTGTCTTATCCCAACACCAC |
| | | | CAGCTTTGGACTCTTTCTTGTGAAACCCGACAACCCATGGGAATGA |
| 35 | muIgGk | Not | ATGGAGACCGACACCTTGCTATTGTGGGTTTTGCTTCTGTGGGTCCCCGGCA |
| | signal | available | GCACTGGGGACACCACATGTCCTCCACCGGTGAGCATCGAGCACGCAGATAT |
| | peptide- | | CAGAGTCAAAAACTATAGCGTGAACTCAAGGGAGAGATATGTGTGCAATTCTG |
| | mu IL-15Ra | | GATTCAAGAGGAAGGCCGGTACCTCTACCCTGATCGAATGTGTTATCAACAA |
| | sushi | | GAACACCAATGTGGCACACTGGACTACACCTTCTCTTAAATGTATACGCGACC |
| | (aa34-103)- | | CTTCCCTTGCCGGTGGATCCGGGGGATCCGGCGGATCCGGAGGGAGCGGA |
| | GSlinker- | | GGGAGTGGGGGTAGTGGGGGAAATTGGATTGACGTGAGGTATGATCTAGAG |
| | IL-15 | | AAGATTGAATCCCTGATTCAATCCATCCATATTGACACAACTCTGTATACCGAC |
| | (aa49-162) | | TCAGACTTCCACCCGTCCTGTAAGGTGACCGCAATGAATTGCTTTCTGTTGGA |
| | | | ACTTCAAGTAATCCTTCATGAATATAGCAACATGACGCTGAACGAGACCGTGA |
| | | | GGAACGTGCTCTACCTGGCGAATTCCACACTTTCTTCCAACAAGAACGTCGC |
| | | | GGAGTCAGGATGTAAGGAGTGCGAAGAGCTGGAGGAAAAGACCTTCACAGA |
| | | | GTTCCTCCAGAGTTTTATTAGGATCGTACAGATGTTCATCAACACATCCTGA |
| 36 | muIL-18 | NM_008360.2 | ATGGCTGCCATGTCAGAAGACTCTTGCGTCAACTTCAAGGAAATGATGTTTAT |
| | WT | | TGACAACACGCTTTACTTTATACCTGAAGAAAATGGAGACCTGGAATCAGACA |
| | | | ACTTTGGCCGACTTCACTGTACAACCGCAGTAATACGGAATATAAATGACCAA |
| | | | GTTCTCTTCGTTGACAAAAGACAGCCTGTGTTCGAGGATATGACTGATATTGA |
| | | | TCAAAGTGCCAGTGAACCCCAGACCAGACTGATAATATACATGTACAAAGACA |
| | | | GTGAAGTAAGAGGACTGGCTGTGACCCTCTCTGTGAAGGATAGTAAAATGTC |
| | | | TACCCTCTCCTGTAAGAACAAGATCATTTCCTTTGAGGAAATGGATCCACCTG |
| | | | AAAATATTGATGATATACAAAGTGATCTCATATTCTTTCAGAAACGTGTTCCAG |
| | | | GACACAACAAGATGGAGTTTGAATCTTCACTGTATGAAGGACACTTTCTTGCT |
| | | | TGCCAAAAGGAAGATGATGCTTTCAAACTCATTCTGAAAAAAAAGGATGAAAA |
| | | | TGGGGATAAATCTGTAATGTTCACTCTCACTAACTTACATCAAAGTTAG |
| 37 | gausia | Not | ATGGGAGTGAAAGTTCTTTTTGCCCTTATTTGTATTGCTGTGGCCGAGGCCAA |
| | signal | available | CTTCGGCCGCCTGCACTGCACTACCGCCGTGATCAGAAACATCAACGATCAG |
| | peptide- | | GTGCTTTTCGTGGATAAGCGCCAGCCCGTGTTTGAGGATATGACTGACATTG |
| | muIL-18 | | ATCAGAGCGCCTCTGAGCCACAGACCCGCCTGATTATCTATATGTATAAGGAC |
| | | | TCCGAAGTCAGAGGCCTTGCGGTTACTCTGTCCGTGAAGGACTCCAAGATGT |
| | | | CTACGCTGTCCTGTAAGAATAAGATTATCAGCTTCGAAGAGATGGACCCTCCA |
| | | | GAGAACATCGACGATATTCAGAGCGATCTTATCTTTTTCCAGAAGCGTGTGCC |
| | | | CGGCCATAACAAGATGGAGTTTGAATCTAGTCTGTATGAGGGACACTTCCTG |
| | | | GCCTGCCAGAAGGAGGACGATGCGTTCAAGCTGATCCTGAAGAAAAAGGAC |
| | | | GAAAACGGCGATAAGAGTGTCATGTTCACCCTGACGAATCTTCACCAATCT |
| 38 | muIL-18 | Not | ATGGCTGCCATGTCAGAAGACTCTTGCGTCAACTTCAAGGAAATGATGTTTAT |
| | mutant | available | TGACAACACGCTTTACTTTATACCTGAAGAAAATGGAGACCTGGAATCAGACC |
| | CS2 | | ATTTTGGCCGACTTCACTGTACAACCGCAGTAATACGGAATATAAATGACCAA |
| | (Decoy | | GTTCTCTTCGTTGACAAAAGACAGCCTGTGTTCGAGGATATGACTGATATTGA |
| | Receptor) | | TCAAAGTGCCAGTGAACCCCAGACCAGACTGATAATATACGCGTACGGCGAC |
| | | | AGTCGCGCCAGAGGAAAAGCTGTGACCCTCTCTGTGAAGGATAGTAAAATGT |
| | | | CTACCCTCTCCTGTAAGAACAAGATCATTTCCTTTGAGGAAATGGATCCACCT |
| | | | GAAAATATTGATGATATACAAAGTGATCTCATATTCTTTCAGAAACGTGTTCCA |
| | | | GGACACAACAAGATGGAGTTTGAATCTTCACTGTATGAAGGACACTTTCTTGC |
| | | | TTGCCAAAAGGAAGATGATGCTTTCAAACTCATTCTGAAAAAAAAGGATGAAA |
| | | | ATGGGGATAAATCTGTAATGTTCACTCTCACTAACTTACATCAAAGTTAG |
| 39 | gausia | Not | ATGGGAGTGAAAGTTCTTTTTGCCCTTATTTGTATTGCTGTGGCCGAGGCCCA |
| | signal | available | CTTCGGCCGCCTGCATTGCACGACCGCAGTTATTCGCAATATTAACGACCAG |
| | peptide- | | GTCCTGTTCGTCGACAAGCGCCAGCCGGTGTTCGAGGACATGACAGACATCG |
| | muIL-18 | | ACCAGTCAGCTAGCGAACCCCAGACAAGATTGATTATCTACGCGTACGGCGA |
| | mutant | | CTCACGCGCTAGGGGCAAAGCCGTGACCCTGAGCGTGAAAGATTCTAAGATG |
| | CS2(Decoy | | AGTACTCTGAGCTGTAAGAACAAGATTATCTCTTTCGAAGAGATGGACCCTCC |
| | Receptor) | | AGAGAACATCGACGATATCCAGAGTGACTTGATTTTTTTCCAGAAGAGAGTGC |
| | | | CCGGACACAACAAGATGGAGTTTGAATCTAGCCTGTATGAGGGCCACTTCCT |
| | | | GGCATGTCAGAAGGAGGATGACGCGTTTAAACTGATCCTGAAGAAAAAGGAC |
| | | | GAAAACGGGGATAAGAGCGTGATGTTCACTCTGACTAACCTCCACCAGTCC |
| 40 | muCCL5 | NM_013653.3 | ATGAAGATCTCTGCAGCTGCCCTCACCATCATCCTCACTGCAGCCGCCCTCT |
| | | | GCACCCCCGCACCTGCCTCACCATATGGCTCGGACACCACTCCCTGCTGCTT |
| | | | TGCCTACCTCTCCCTCGCGCTGCCTCGTGCCCACGTCAAGGAGTATTTCTAC |
| | | | ACCAGCAGCAAGTGCTCCAATCTTGCAGTCGTGTTTGTCACTCGAAGGAACC |
| | | | GCCAAGTGTGTGCCAACCCAGAGAAGAAGTGGGTTCAAGAATACATCAACTA |
| | | | TTTGGAGATGAGCTAG |
| 41 | muCCL19 | NM_011888.4 | ATGGCCCCCCGTGTGACCCCACTCCTGGCCTTCAGCCTGCTGGTTCTCTGGA |
| | | | CCTTCCCAGCCCCAACTCTGGGGGGTGCTAATGATGCGGAAGACTGCTGCCT |
| | | | GTCTGTGACCCAGCGCCCCATCCCTGGGAACATCGTGAAAGCCTTCCGCTAC |
| | | | CTTCTTAATGAAGATGGCTGCAGGGTGCCTGCTGTTGTGTTCACCACACTAAG |
| | | | GGGCTATCAGCTCTGTGCACCTCCAGACCAGCCCTGGGTGGATCGCATCATC |
| | | | CGAAGACTGAAGAAGTCTTCTGCCAAGAACAAAGGCAACAGCACCAGAAGGA |
| | | | GCCCTGTGTCTTGA |
| 42 | muCXCL9 | NM_008599.4 | ATGAAGTCCGCTGTTCTTTTCCTCTTGGGCATCATCTTCCTGGAGCAGTGTGG |
| | | | AGTTCGAGGAACCCTAGTGATAAGGAATGCACGATGCTCCTGCATCAGCACC |
| | | | AGCCGAGGCACGATCCACTACAAATCCCTCAAAGACCTCAAACAGTTTGCCC |
| | | | CAAGCCCCAATTGCAACAAAACTGAAATCATTGCTACACTGAAGAACGGAGAT |
| | | | CAAACCTGCCTAGATCCGGACTCGGCAAATGTGAAGAAGCTGATGAAAGAAT |
| | | | GGGAAAAGAAGATCAGCCAAAAGAAAAAGCAAAAGAGGGGGAAAAAACATCA |
| | | | AAAGAACATGAAAAACAGAAAACCCAAAACACCCCAAAGTCGTCGTCGTTCAA |
| | | | GGAAGACTACATAA |
| 43 | muXCL1 | NM_008510.2 | ATGAGACTTCTCCTCCTGACTTTCCTGGGAGTCTGCTGCCTCACCCCATGGG |
| | | | TTGTGGAAGGTGTGGGGACTGAAGTCCTAGAAGAGAGTAGCTGTGTGAACTT |
| | | | ACAAACCCAGCGGCTGCCAGTTCAAAAAATCAAGACCTATATCATCTGGGAG |
| | | | GGGGCCATGAGAGCTGTAATTTTTGTCACCAAACGAGGACTAAAAATTTGTGC |
| | | | TGATCCAGAAGCCAAATGGGTGAAAGCAGCGATCAAGACTGTGGATGGCAGG |
| | | | GCCAGTACCAGAAAGAACATGGCTGAAACTGTTCCCACAGGAGCCCAGAGGT |
| | | | CCACCAGCACAGCGATAACCCTGACTGGGTAA |
| 44 | muXCL1- | Not | ATGAGACTGCTGCTGCTGACATTCCTGGGCGTGTGCTGTCTGACACCCTGGG |
| | V21C/A59C | available | TTGTCGAAGGCGTGGGAACAGAGGTGCTGGAAGAGTCCAGCTGCGTGAATC |
| | | | TGCAGACCCAGAGACTGCCCTGCCAGAAGATCAAGACCTACATCATCTGGGA |
| | | | GGGCGCCATGAGAGCCGTGATCTTCGTGACAAAGAGAGGCCTGAAGATCTG |
| | | | CGCCGATCCTGAGGCCAAATGGGTCAAAGCCTGCATCAAGACCGTGGACGG |
| | | | CAGAGCCAGCACCAGAAAGAACATGGCCGAGACAGTGCCTACAGGCGCCCA |
| | | | GAGATCTACCAGCACAGCCATCACACTGACCGGCTAA |
| 45 | muIL-28A | NM_001024673.2 | ATGCTCCTCCTGCTGTTGCCTCTGCTGCTGGCCGCAGTGCTGACAAGAACCC |
| | (Interferon | | AAGCTGACCCTGTCCCCAGGGCCACCAGGCTCCCAGTGGAAGCAAAGGATT |
| | lambda 2) | | GCCACATTGCTCAGTTCAAGTCTCTGTCCCCAAAAGAGCTGCAGGCCTTCAAA |
| | | | AAGGCCAAGGATGCCATCGAGAAGAGGCTGCTTGAGAAGGACCTGAGGTGC |
| | | | AGTTCCCACCTCTTCCCCAGGGCCTGGGACCTGAAGCAGCTGCAGGTCCAA |
| | | | GAGCGCCCCAAGGCCTTGCAGGCTGAGGTGGCCCTGACCCTGAAGGTCTGG |
| | | | GAGAACATGACTGACTCAGCCCTGGCCACCATCCTGGGCCAGCCTCTTCATA |
| | | | CACTGAGCCACATTCACTCCCAGCTGCAGACCTGTACACAGCTTCAGGCCAC |
| | | | AGCAGAGCCCAGGTCCCCGAGCCGCCGCCTCTCCCGCTGGCTGCACAGGCT |
| | | | CCAGGAGGCCCAGAGCAAGGAGACCCCTGGCTGCCTGGAGGCCTCTGTCAC |
| | | | CTCCAACCTGTTTCGCCTGCTCACCCGGGACCTCAAGTGTGTGGCCAATGGA |
| | | | GACCAGTGTGTCTG |
| 46 | muIL-28B | NM_177396.1 | ATGCTCCTCCTGCTGTTGCCTCTGCTGCTGGCCGCAGTGCTGACAAGAACCC |
| | (Interferon | | AAGCTGACCCTGTCCCCAGGGCCACCAGGCTCCCAGTGGAAGCAAAGGATT |
| | lambda 3) | | GCCACATTGCTCAGTTCAAGTCTCTGTCCCCAAAAGAGCTGCAGGCCTTCAAA |
| | | | AAGGCCAAGGGTGCCATCGAGAAGAGGCTGCTTGAGAAGGACATGAGGTGC |
| | | | AGTTCCCACCTCATCTCCAGGGCCTGGGACCTGAAGCAGCTGCAGGTCCAAG |
| | | | AGCGCCCCAAGGCCTTGCAGGCTGAGGTGGCCCTGACCCTGAAGGTCTGGG |
| | | | AGAACATAAATGACTCAGCCCTGACCACCATCCTGGGCCAGCCTCTTCATACA |
| | | | CTGAGCCACATTCACTCCCAGCTGCAGACCTGTACACAGCTTCAGGCCACAG |
| | | | CAGAGCCCAAGCCCCCGAGTCGCCGCCTCTCCCGCTGGCTGCACAGGCTCC |
| | | | AGGAGGCCCAGAGCAAGGAGACTCCTGGCTGCCTGGAGGACTCTGTCACCT |
| | | | CCAACCTGTTTCAACTGCTCCTCCGGGACCTCAAGTGTGTGGCCAGTGGAGA |
| | | | CCAGTGTGTCTGA |

### Examples

The invention will now be illustrated by means of the following synthetic and biological examples, which do not limit the scope of the invention in any way.

### Materials and Methods:

mRNA in vitro transcription mRNAs encoding various cytokines (mulL-21, mulL-7 and mulL-15sushi), co-stimulatory molecules (mu4-1BBL) or FireFly Luciferase or NanoLuciferase were prepared in vitro by T7-mediated transcription from linearized DNA templates (peTheRNAvs3 vector), which incorporates 5′ and 3′ UTRs and a polyA tail. The final mRNA utilizes Cap1 and 100% replacement of uridine with N1-methyl-pseudo-uridine.

mRNA Lipid-Based Nanoparticle Synthesis and Purification

Lipid-based nanoparticles are produced by microfluidic mixing of an mRNA solution in sodium acetate buffer (100 mM, pH4) and lipid solution in a 2:1 volume ratio at a speed of 9 mL/min using the NanoAssemblr Benchtop (Precision Nanosystems). The lipid solution contained a mixture of a suited ionizable lipid, DSPC (Avanti), Cholesterol (Sigma) and DMG-PEG2000 (Sunbright GM-020, NOF corporation). The 4 lipids were mixed at standard ratio 50/10/38.5/1.5 (ionizable lipid, helper lipid, Cholesterol, PEG lipid). LNPs were dialyzed against TBS (10000 times more TBS volume than LNP volume) using slide-a-lyzer dialysis cassettes (20K MWCO, 3 mL, ThermoFisher). Size, polydispersity and zeta potential were measured with a Zetasizer Nano (Malvern). mRNA encapsulation was measured by standard Ribogreen RNA assay (Invitrogen).

### Cell Lines:

The most optimal culturing conditions including growth medium, subcultivation ratio, and medium renewal recommendations are summarized below. To harvest adherent cells, used-up growth medium was discarded and cells were rinsed twice with phosphate buffered saline (PBS) (Sigma) before addition of trypsine-EDTA (0.05%) (Gibco, Thermo Fisher Scientific) to loosen the cells. Medium renewal needs to occur every 2 to 3 days, whenever cells reached confluency of approximately 70%. Cell viability was determined using the Vi-Cell XR Cell Viability Analyzer (Beckman Coulter).

TABLE 4
Cell type specific culturing conditions

| Cell | | | Growth | Subcul- |
|---|---|---|---|---|
| type | Description | Source | Medium | tivation |
| MC38 | Adherent | Kerafast | DMEM high Glucose + | 1:4 to 1:10 |
| | mouse | | 2 mM L-glutamin + | |
| | colon | | 100 units | |
| | fibroblast | | Penicilin + + 40.1 | |
| | | | mg/ml steptomycin | |
| | | | (all from Sigma) + | |
| | | | 10% FBS (PAN) | |

Abbreviations:
DMEM: Dulbecco's Modified Eagle Medium;
FBS: Foetal Bovine Serum

### Tumor Inoculation and Intra-Tumoral Injection

MC38 tumor cells were inoculated in C57/BL6 mice. Female C57BL/6J Mice were purchased from Charles River Laboratories (France) and housed in individually ventilated cages containing standard bedding material. The animals were maintained and treated in accordance with the institutional (UGhent) and European Union guidelines for animal experimentation. Mice had ad libitum access to food and water. Experiments started when mice were approx. 7 to 9 weeks old. To prepare for subcutaneous tumor inoculation, mice were anesthetized using 2.5% isoflurane and the injection site was shaved. The injection site is typically on the posterior/lateral aspect of the lower left flank. For inoculation purposes, cells need to be approximately 1 week in culture and between passage 3 and 5 after thawing. Cold tumor cell solution was injected subcutaneously into the left flank at a dose of 0.5*10e6 cells/50 μl PBS. Tumor growth was measured every 2-3 days using the Caliper device. The following formula was used to calculate tumor size: (tumor width*tumor width*tumor length)/2. When tumors reached a mean volume of 50-100 mm³, mice were randomized in vehicle- and mRNA-treated groups (8-10 mice per group) and treatments were initiated. Tumor were injected as a monotherapy (or combination therapy) with LNPs containing mRNA (5 to 15 μg mRNA dose in 20 μl TBS buffer) or with control buffer (TBS) using a U-100 insulin needle (BD Biosciences, San Diego, CA, USA). After injections, mice were always monitored for 5-10 minutes until fully awake without showing any sings of pain distress or complications. Mice were monitored every other day and tumor volumes were measured with calipers and body weight followed 2-3 times a week after treatment

For combination therapies, mRNAs encoding different cytokines were also formulated in s-Ac7-Dog LNPs. Animals were treated with 3 intratumoral doses of mRNAs (10 μg or 15 μg total mRNA/dose; as indicated) when the tumor volume reached 50-75 mm³. Doses were injected at day of randomization (DR), DR+3 and DR+7. Control animals were treated with an equivalent dose of irrelevant mRNA (NanoLuc). Tumor volume was measured at the indicated time points using calipers and was recorded in cubic millimeters. The effect of a combination therapy was tested for the combination of IL-21 with other immunomodulatory molecules such as II-7, IL-15sushi, 4-1BBL.

### Data Analysis

All raw data were analyzed using the Graph Pad Prism version 7 software.

### Results

### Example 1—mRNA Monotherapy Efficacy in a MC38 (Colon Cancer) Model

Monotherapy efficacy using IL-21 mRNA monotherapy was assessed in a MC38 colon adenocarcinoma cancer model. 5×10⁵ MC-38 colon tumor cells were established subcutaneously in C57BL/6 mice. mRNAs encoding IL-21 and Fluc as irrelevant molecule were prepared as described in M&M and formulated in LNPs. Animals were treated with 3 intratumoral doses of mRNAs (15 μg total mRNA/dose) when tumor volume reached 50-75 mm³. Doses were injected at day of randomization (DR), DR+3 and DR+7. Control animals were treated with an equivalent dose of negative control mRNA. Tumor volume was measured at the indicated time points using calipers and was recorded in cubic millimeters.

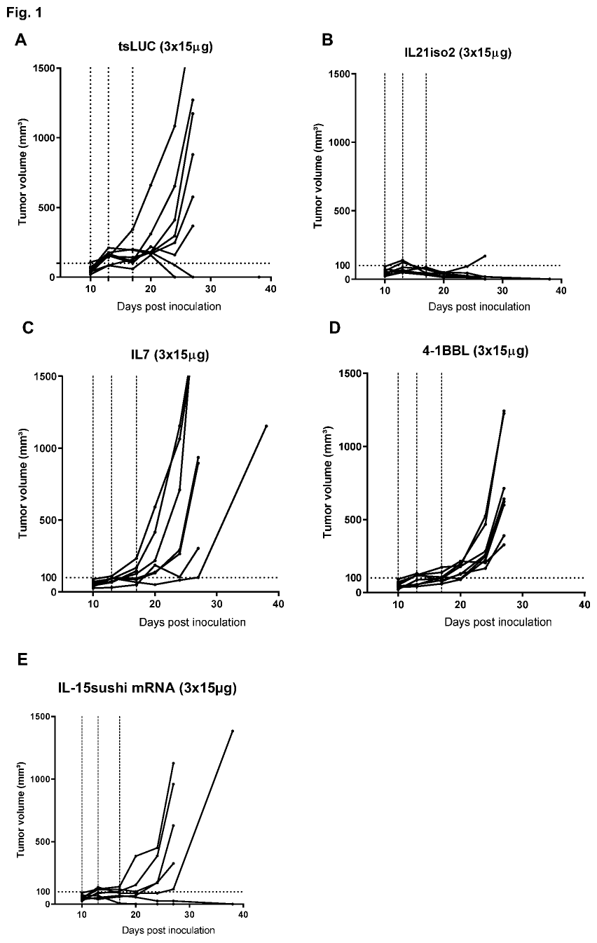

A complete response (“CR”) was observed in 2 of 8 subjects (20%) treated with Fluc mRNA control (3×15 μg total mRNA/dose) (FIG. 1A). Treatment with IL-21 mRNA (3×15 μg total mRNA/dose) elicited a complete response in 6 of 8 subjects (75%) (FIG. 1B). FIG. 1C, FIG. 1D and FIG. 1E show a lack of efficacy for respectively IL-7, 4-1BBL or IL-15sushi mRNA monotherapy treatment (3×15 μg total mRNA/dose). A complete response (“CR”) was observed in 2 of 8 subjects (20%) treated with IL-15sushi, similar to the control group (FIG. 1E).

### Example 2—Synergistic Efficacy of a Combination mRNA Therapy in a MC38 (Colon Cancer) Model IL-7 and IL-21

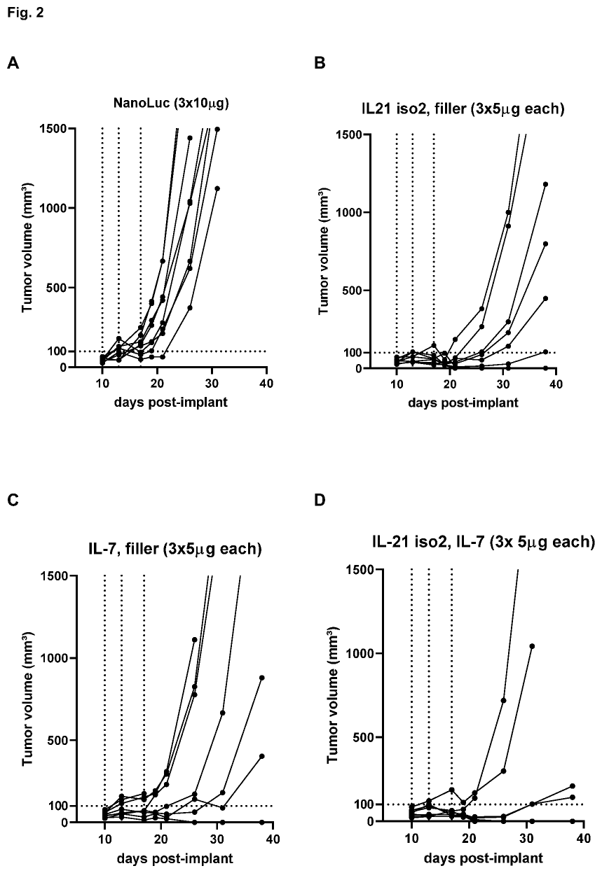

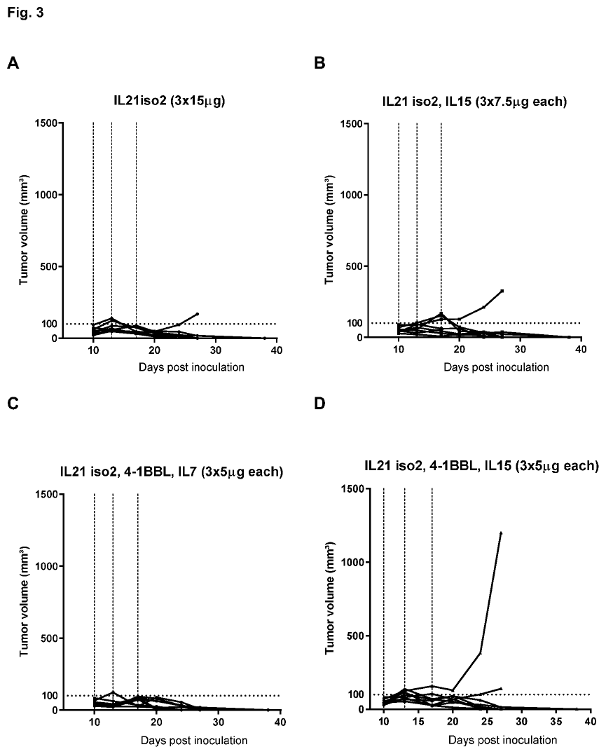

Addition of IL-7 to IL-21-encoding mRNA increases the efficacy of the treatment in the MC38 colon cancer model. Treatment with NanoLuc mRNA control (3×10 μg mRNA) showed a lack of efficacy (FIG. 2A). Treatment with mRNA encoding IL-21 monotherapy (3×5 μg total mRNA/dose) and filler RNA NanoLuc (3×5 μg total mRNA/dose) showed a complete response (“CR”) in 2 of 8 subjects (20%) and a partial response in 2 out of 8 animals (20%) (FIG. 2B). Treatment with mRNA encoding IL-7 monotherapy (3×5 μg total mRNA/dose) and filler RNA NanoLuc (3×5 μg total mRNA/dose) showed a complete response (“CR”) in 1 of 8 subjects (20%) and a partial response in 2 out of 8 animals (20%) (FIG. 2C). A combination therapy with mRNA encoding IL-7 and IL-21 (3×5 μg each; 10 μg total mRNA/dose) resulted in a complete response (“CR”) in 3 of 8 subjects (37.5%) and a partial response in 2 out of 8 animals (20%) (FIG. 2D). The data showed that IL-21/IL-7 combination led to improved efficacy when compared to the monotherapy treatment.

### IL-15Sushi and IL-21

Treatment with mRNA encoding IL-21 and IL-15sushi mRNA (3×7.5 μg each; 15 μg total mRNA/dose) does not seem to increase the efficacy of the treatment in the MC38 colon cancer model which is observed with IL-21 alone (resp. FIG. 3B and FIG. 3A). In both treatment therapies, complete response was observed in 6 of 8 animals (75%).

### IL-7, 4-1BBL and IL-21

Combining mRNAs encoding IL-21, IL-7 and 4-1BBL (3×5 μg each mRNA/dose) resulted in a 100% response in all animals (FIG. 3C).

### IL-15sushi, 4-1BBL and IL-21

Combining mRNAs encoding IL-21, IL-15sushi and 4-1BBL (3×5 μg each mRNA/dose) resulted in a complete response in 6 of 8 animals (75%) (FIG. 3D).

### Example 3—Generation of Systemic Memory Immune Response after Treatment with IL-21 Monotherapy or IL-21-Based Mix Therapy

Memory immune response in animals treated with IL-21 monotherapy therapy or combinational API therapy was evaluated. Mice inoculated with 5×10⁵ MC38 tumor cells were treated with IL-21 monotherapy. Fifteen micrograms of total mRNA were injected intratumorally (3 times) as described in previous example. At day 80 post tumor inoculation, complete responders from the treatment group were rechallenged with 5×10⁵ MC-38 colon tumors in the opposite flank. As control, 12 naive animals were also injected with the MC38 cancer.

After Treatment with IL-21 Monotherapy

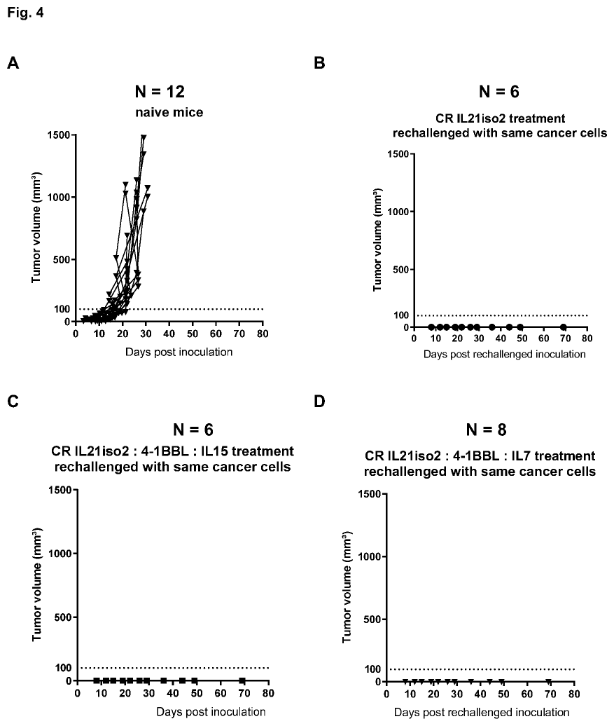

Naïve animals (n=12) were not able to control tumor growth (FIG. 4A). In contrast, complete responder animals (n=6) that received intratumoral treatment were rechallenged with the same cancer cells, no tumoral growth was observed in any of the rechallenged mice at least until day 22 post-inoculation (6 out of 6 animals) (FIG. 4B). The generation of systemic anti-cancer memory post local therapy was observed for mice treated with monotherapy IL-21.

### After Combinatorial API Treatment

Complete responder animals that received intratumoral treatment were rechallenged with the same cancer cells, no tumoral growth was observed in any of the rechallenged mice at least until day 26 post-inoculation: 6 out of 6 animals in IL-21/4-1BBL/IL-15sushi treatment group (FIG. 4C) and 8 out of 8 animals in IL-21/4-1BBL/IL-7 treatment group (FIG. 4D). These results show that intratumoral injection of mRNA encoding IL-21/4-1BBL/IL-15sushi or IL-21/4-1BBL/IL-7 induce memory immune response with anti-tumoral effects.

### Example 4—Synergistic Efficacy of IL-21 with IL-7 and 4-1BBL mRNA Therapy at 10 μg Dose in a MC38 (Colon Cancer) Model

Monotherapy versus combinational therapy of 2 or 3 mRNA was assessed in a MC38 colon adenocarcinoma cancer model. 5×10⁵ MC-38 colon tumor cells were established subcutaneously in C57BL/6 mice. mRNAs encoding ntrIL-21 (non-translated IL-21), IL-21, IL-7 and 4-1BBL were prepared as described in M&M and formulated in LNPs. Animals were treated with 3 intratumoral doses of mRNAs (3.33 μg mRNA for 1 API per dose, 6.66 μg mRNA for 2 API per dose and 10 μg total mRNA for 3 API per dose) when tumor volume reached 50-75 mm³. Doses were injected at day of randomization (DR), DR+3 and DR+7. Control animals were treated with an equivalent dose of non-translated IL-21 mRNA. Tumor volume was measured at the indicated time points using calipers and was recorded in cubic millimeters.

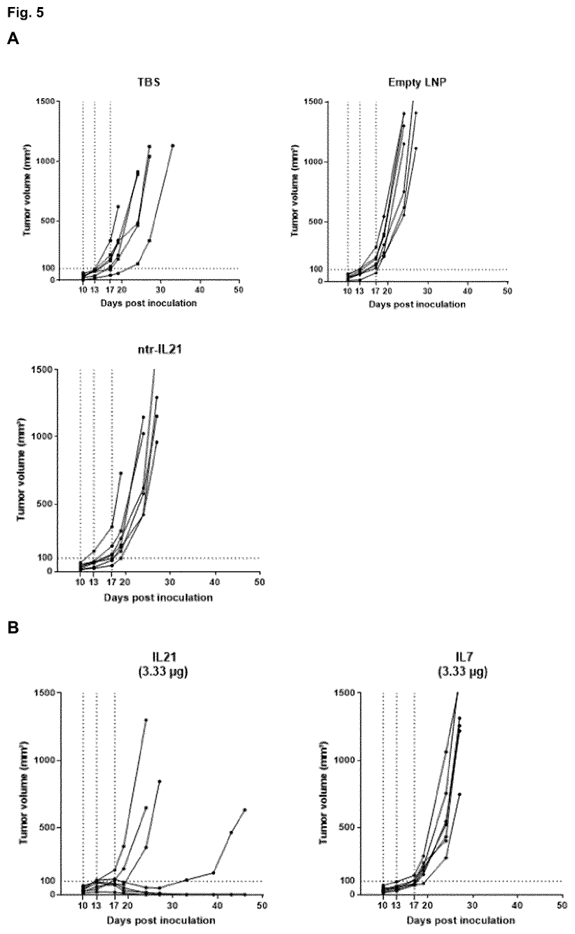

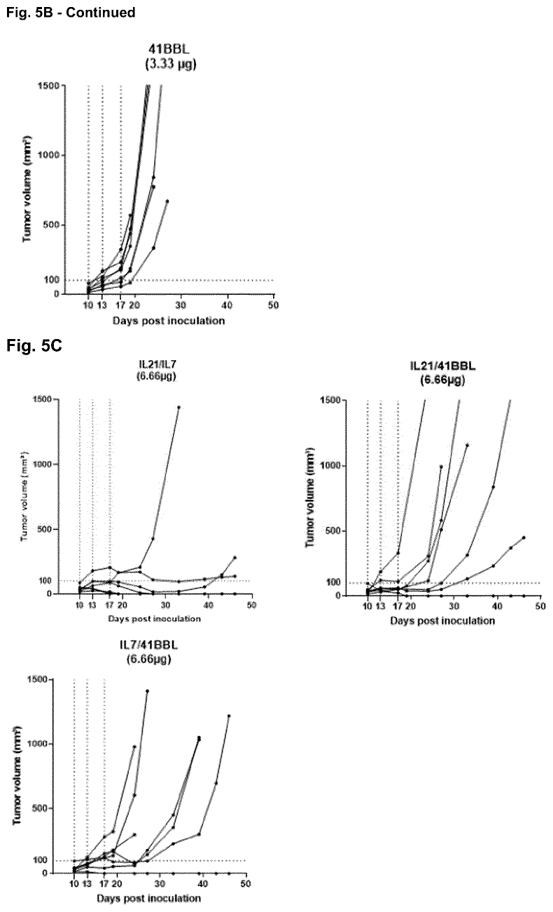

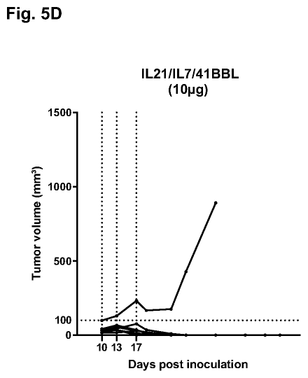

Treatment with IL-21 mRNA (3×3.3 μg total mRNA/dose) elicited a complete response (“CR”) in 3 of 7 subjects (42%) and a partial response in 1 out of 7 animals (14%) (FIG. 5B). IL-7 or 4-1BBL mRNA monotherapy treatment (3×3.33 μg total mRNA/dose) showed a lack of efficacy as for TBS, empty LNP and non-translated IL-21 mRNA LNP treated animals (FIG. 5A).

A combination therapy with mRNA encoding IL-21 and IL-7 (3×3.33 μg each; 6.66 μg total mRNA/dose) resulted in a complete response in 4 of 7 subjects (57%) and a partial response in 2 out of 7 animals (28%) (FIG. 5C). The data showed that IL-21/IL-7 combination led to improved efficacy when compared to the monotherapy treatment. Combining mRNA encoding IL-21 and 4-1BBL or mRNA encoding IL-7 and 4-1BBL does not seem to significantly increase the efficacy of the treatment in the MC38 colon cancer model with one CR out of 7 (14%) (FIG. 5C).

Combining mRNAs encoding all 3 molecules IL-21, IL-7 and 4-1BBL (3×3.33 μg each mRNA/dose, 10 μg total mRNA/dose) resulted in an 85% response rate (FIG. 5D).

### REFERENCES

Kariko, K., Muramatsu, H., Ludwig, J. & Weissman, D. Generating the optimal mRNA for therapy: HPLC purification eliminates immune activation and improves translation of nucleoside-modified, protein-encoding mRNA. Nucleic Acids Res. 39, e142 (2011). This study demonstrates the importance of purification of IVT mRNA in achieving potent protein translation and in suppressing inflammatory responses.

1. A composition comprising one or more isolated non-viral nucleic acid molecule(s) encoding the functional immunostimulatory protein IL-21 and encoding a co-stimulatory molecule.

2. The composition as defined in claim 1, wherein said co-stimulatory molecule is selected from the list comprising 4-1BBL, OX40L, ICOS ligand, and CD40L; in particular 4-1BBL.

3. The composition as defined in claim 1 or 2; further comprising one or more nucleic acid molecules encoding a cytokine and/or a chemokine.

4. The composition as defined in claim 3, wherein:
said cytokine is selected from the list comprising IL-7, IL-12, IL-18, IFNlambda, IL-15sushi, IL-23, a type I IFN, a type II IFN, a type III IFN; and/or
said chemokine is selected from the list comprising CCL19, CCL21, CXCL9, CXCL10, CCL5 and XCL1.

5. The composition as defined in any one of claim 1 or 4; further comprising one or more of: a target-specific antigen, and/or a checkpoint inhibitor.

6. The composition as defined in claim 5; wherein said target-specific antigen is a tumor-associated antigen or a neoantigen.

7. The composition as defined in claim 5 wherein said checkpoint inhibitor is selected from the list comprising inhibitory molecules of: PD-1, PD-L1, CTLA4, TIGIT, TIM3, LAG6 and VISTA; preferably inhibitory molecules of PD-1, PD-L1, or CTLA4.

8. The composition as defined in claim 1; comprising one or more isolated non-viral nucleic acid molecule(s) encoding any of the following combinations:
IL-21, 4-1BBL and IL-7;
IL-21, 4-1BBL and IL-15sushi;
IL-21, 4-1BBL and IL-18; or
IL-21, 4-1BBL and IFNlambda.

9. The composition as defined in any one of claims 1-8, wherein said one or more nucleic acid molecule(s) are linear or circular nucleic acids.

10. The composition as defined in any one of claims 1-9, wherein said one or more nucleic acid molecule(s) are selected from the group comprising RNA and DNA, preferably RNA, more preferably mRNA.

11. The composition as defined in any one of claims 1-10, wherein said one or more nucleic acid molecule(s) comprise(s) one or more of the following: a 5′CAP, a poly(A) tail and/or modified nucleoside(s); preferably said modified nucleoside is N1-methylpseudouridine.

12. The composition as defined in anyone of claims 1-11; wherein said one or more nucleic acid molecule(s) is/are in the form of naked nucleic acid molecule(s) or nucleic acid(s) encapsulated in nanoparticles, such as lipid nanoparticles or polymeric nanoparticle, in particular lipid nanoparticles.

13. A lipid nanoparticle comprising the composition as defined in any one of claims 1-12.

14. A pharmaceutical formulation comprising the composition as defined in any one of claims 1-12, or the lipid nanoparticle as defined in claim 13 and at least one pharmaceutically acceptable carrier or excipient.

15. The composition as defined in any one of claims 1-12; the lipid nanoparticle as defined in claim 13 or the pharmaceutical formulation as defined in claim 14; for use in parenteral administration; more in particular for use in intravenous, intratumoral, intradermal, intraperitoneal, intramuscular or intranodal administration, preferably intratumoral administration.

16. The composition as defined in any one of claims 1-12; the lipid nanoparticle as defined in claim 13 or the pharmaceutical formulation as defined in claim 14; for use in human or veterinary medicine.

17. The composition as defined in any one of claims 1-12; the lipid nanoparticle as defined in claim 13 or the pharmaceutical formulation as defined in claim 14; for use in combination with standard of care cancer therapies including radiotherapy, targeted therapy, and chemotherapy.

18. The composition as defined in any one of claims 1-12; the lipid nanoparticle as defined in claim 13 or the pharmaceutical formulation as defined in claim 14; for use in the prevention and/or treatment of cell proliferative disorders.


SEQUENCE LISTING (46 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 153 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKVSTLSFI

SEQ ID NO: 3 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - protein, 133 aa, Homo sapiens
MFHVSFRYIFGLPPLILVLLPVASSDCDIEGKDGKQYESVLMVSIDQLLDSMKEIGSNCLNNEFNFFKRHICDANKVKGRKPAALGEAQPTKSLEENKSLKEQKKLNDLCFLKRLLQEIKTCWNKILMGTKEH

SEQ ID NO: 5 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 7 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 8 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 9 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 10 - protein, 114 aa, Homo sapiens
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 11 - protein, 114 aa, Homo sapiens
MRLLILALLGICSLTAYIVEGVGSEVSDKRTCVSLTTQRLPCSRIKTYTITEGSLRAVIFITKRGLKVCADPQATWVRDCVRSMDRKSNTRNNMIQTKPTGTQQSTNTAVTLTG

SEQ ID NO: 12 - protein, 200 aa, Homo sapiens
MAAAWTVVLVTLVLGLAVAGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 13 - protein, 200 aa, Homo sapiens
MKLDMTGDCTPVLVLMAAVLTVTGAVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 14 - protein, 196 aa, Homo sapiens
MTGDCMPVLVLMAAVLTVTGAVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 15 - protein, 162 aa, Mus musculus
MERTLVCLVVIFLGTVAHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKVCTLNAFLSLPCCVRVPPVPSDS

SEQ ID NO: 16 - protein, 146 aa, Mus musculus [sequence omitted]

SEQ ID NO: 17 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 18 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 19 - protein, 225 aa, Mus musculus
METDTLLLWVLLLWVPGSTGDTTCPPPVSIEHADIRVKNYSVNSRERYVCNSGFKRKAGTSTLIECVINKNTNVAHWTTPSLKCIRDPSLAGGSGGSGGSGGSGGSGGSGGNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 20 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 21 - protein, 174 aa, Mus musculus
MGVKVLFALICIAVAEANFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 22 - protein, 192 aa, Mus musculus
MAAMSEDSCVNFKEMMFIDNTLYFIPEENGDLESDHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 23 - protein, 174 aa, Mus musculus
MGVKVLFALICIAVAEAHFGRLHCTTAVIRNINDQVLFVDKRQPVFEDMTDIDQSASEPQTRLIIYAYGDSRARGKAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 24 - protein, 91 aa, Mus musculus
MKISAAALTIILTAAALCTPAPASPYGSDTTPCCFAYLSLALPRAHVKEYFYTSSKCSNLAVVFVTRRNRQVCANPEKKWVQEYINYLEMS

SEQ ID NO: 25 - protein, 108 aa, Mus musculus
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 26 - protein, 126 aa, Mus musculus
MKSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTT

SEQ ID NO: 27 - protein, 114 aa, Mus musculus
MRLLLLTFLGVCCLTPWVVEGVGTEVLEESSCVNLQTQRLPVQKIKTYIIWEGAMRAVIFVTKRGLKICADPEAKWVKAAIKTVDGRASTRKNMAETVPTGAQRSTSTAITLTG

SEQ ID NO: 28 - protein, 114 aa, Mus musculus
MRLLLLTFLGVCCLTPWVVEGVGTEVLEESSCVNLQTQRLPCQKIKTYIIWEGAMRAVIFVTKRGLKICADPEAKWVKACIKTVDGRASTRKNMAETVPTGAQRSTSTAITLTG

SEQ ID NO: 29 - protein, 193 aa, Mus musculus
MLLLLLPLLLAAVLTRTQADPVPRATRLPVEAKDCHIAQFKSLSPKELQAFKKAKDAIEKRLLEKDLRCSSHLFPRAWDLKQLQVQERPKALQAEVALTLKVWENMTDSALATILGQPLHTLSHIHSQLQTCTQLQATAEPRSPSRRLSRWLHRLQEAQSKETPGCLEASVTSNLFRLLTRDLKCVANGDQCV

SEQ ID NO: 30 - protein, 193 aa, Mus musculus
MLLLLLPLLLAAVLTRTQADPVPRATRLPVEAKDCHIAQFKSLSPKELQAFKKAKGAIEKRLLEKDMRCSSHLISRAWDLKQLQVQERPKALQAEVALTLKVWENINDSALTTILGQPLHTLSHIHSQLQTCTQLQATAEPKPPSRRLSRWLHRLQEAQSKETPGCLEDSVTSNLFQLLLRDLKCVASGDQCV

SEQ ID NO: 31 - DNA, 489 bp, Mus musculus
  source 1..489: mol_type = other DNA
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaaggtatgcaccttaaatgcatttctttcacttccatgttgtgtccgggtacctcctgtgcccagtgactcatag

SEQ ID NO: 32 - DNA, 441 bp, Mus musculus
  source 1..441: mol_type = other DNA
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcctag

SEQ ID NO: 33 - DNA, 465 bp, Mus musculus
  source 1..465: mol_type = other DNA
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataa

SEQ ID NO: 34 - DNA, 930 bp, Mus musculus
  source 1..930: mol_type = other DNA
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggttttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaatga

SEQ ID NO: 35 - DNA, 678 bp, Mus musculus
  source 1..678: mol_type = other DNA
atggagaccgacaccttgctattgtgggttttgcttctgtgggtccccggcagcactggggacaccacatgtcctccaccggtgagcatcgagcacgcagatatcagagtcaaaaactatagcgtgaactcaagggagagatatgtgtgcaattctggattcaagaggaaggccggtacctctaccctgatcgaatgtgttatcaacaagaacaccaatgtggcacactggactacaccttctcttaaatgtatacgcgacccttcccttgccggtggatccgggggatccggcggatccggagggagcggagggagtgggggtagtgggggaaattggattgacgtgaggtatgatctagagaagattgaatccctgattcaatccatccatattgacacaactctgtataccgactcagacttccacccgtcctgtaaggtgaccgcaatgaattgctttctgttggaacttcaagtaatccttcatgaatatagcaacatgacgctgaacgagaccgtgaggaacgtgctctacctggcgaattccacactttcttccaacaagaacgtcgcggagtcaggatgtaaggagtgcgaagagctggaggaaaagaccttcacagagttcctccagagttttattaggatcgtacagatgttcatcaacacatcctga

SEQ ID NO: 36 - DNA, 579 bp, Mus musculus
  source 1..579: mol_type = other DNA
atggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttag

SEQ ID NO: 37 - DNA, 522 bp, Mus musculus
  source 1..522: mol_type = other DNA
atgggagtgaaagttctttttgcccttatttgtattgctgtggccgaggccaacttcggccgcctgcactgcactaccgccgtgatcagaaacatcaacgatcaggtgcttttcgtggataagcgccagcccgtgtttgaggatatgactgacattgatcagagcgcctctgagccacagacccgcctgattatctatatgtataaggactccgaagtcagaggccttgcggttactctgtccgtgaaggactccaagatgtctacgctgtcctgtaagaataagattatcagcttcgaagagatggaccctccagagaacatcgacgatattcagagcgatcttatctttttccagaagcgtgtgcccggccataacaagatggagtttgaatctagtctgtatgagggacacttcctggcctgccagaaggaggacgatgcgttcaagctgatcctgaagaaaaaggacgaaaacggcgataagagtgtcatgttcaccctgacgaatcttcaccaatct

SEQ ID NO: 38 - DNA, 579 bp, Mus musculus
  source 1..579: mol_type = other DNA
atggctgccatgtcagaagactcttgcgtcaacttcaaggaaatgatgtttattgacaacacgctttactttatacctgaagaaaatggagacctggaatcagaccattttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacgcgtacggcgacagtcgcgccagaggaaaagctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttag

SEQ ID NO: 39 - DNA, 522 bp, Mus musculus
  source 1..522: mol_type = other DNA
atgggagtgaaagttctttttgcccttatttgtattgctgtggccgaggcccacttcggccgcctgcattgcacgaccgcagttattcgcaatattaacgaccaggtcctgttcgtcgacaagcgccagccggtgttcgaggacatgacagacatcgaccagtcagctagcgaaccccagacaagattgattatctacgcgtacggcgactcacgcgctaggggcaaagccgtgaccctgagcgtgaaagattctaagatgagtactctgagctgtaagaacaagattatctctttcgaagagatggaccctccagagaacatcgacgatatccagagtgacttgatttttttccagaagagagtgcccggacacaacaagatggagtttgaatctagcctgtatgagggccacttcctggcatgtcagaaggaggatgacgcgtttaaactgatcctgaagaaaaaggacgaaaacggggataagagcgtgatgttcactctgactaacctccaccagtcc

SEQ ID NO: 40 - DNA, 276 bp, Mus musculus
  source 1..276: mol_type = other DNA
atgaagatctctgcagctgccctcaccatcatcctcactgcagccgccctctgcacccccgcacctgcctcaccatatggctcggacaccactccctgctgctttgcctacctctccctcgcgctgcctcgtgcccacgtcaaggagtatttctacaccagcagcaagtgctccaatcttgcagtcgtgtttgtcactcgaaggaaccgccaagtgtgtgccaacccagagaagaagtgggttcaagaatacatcaactatttggagatgagctag

SEQ ID NO: 41 - DNA, 327 bp, Mus musculus
  source 1..327: mol_type = other DNA
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctccagaccagccctgggtggatcgcatcatccgaagactgaagaagtcttctgccaagaacaaaggcaacagcaccagaaggagccctgtgtcttga

SEQ ID NO: 42 - DNA, 381 bp, Mus musculus
  source 1..381: mol_type = other DNA
atgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacataa

SEQ ID NO: 43 - DNA, 345 bp, Mus musculus
  source 1..345: mol_type = other DNA
atgagacttctcctcctgactttcctgggagtctgctgcctcaccccatgggttgtggaaggtgtggggactgaagtcctagaagagagtagctgtgtgaacttacaaacccagcggctgccagttcaaaaaatcaagacctatatcatctgggagggggccatgagagctgtaatttttgtcaccaaacgaggactaaaaatttgtgctgatccagaagccaaatgggtgaaagcagcgatcaagactgtggatggcagggccagtaccagaaagaacatggctgaaactgttcccacaggagcccagaggtccaccagcacagcgataaccctgactgggtaa

SEQ ID NO: 44 - DNA, 345 bp, Mus musculus
  source 1..345: mol_type = other DNA
atgagactgctgctgctgacattcctgggcgtgtgctgtctgacaccctgggttgtcgaaggcgtgggaacagaggtgctggaagagtccagctgcgtgaatctgcagacccagagactgccctgccagaagatcaagacctacatcatctgggagggcgccatgagagccgtgatcttcgtgacaaagagaggcctgaagatctgcgccgatcctgaggccaaatgggtcaaagcctgcatcaagaccgtggacggcagagccagcaccagaaagaacatggccgagacagtgcctacaggcgcccagagatctaccagcacagccatcacactgaccggctaa

SEQ ID NO: 45 - DNA, 581 bp, Mus musculus
  source 1..581: mol_type = other DNA
atgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaaggatgccatcgagaagaggctgcttgagaaggacctgaggtgcagttcccacctcttccccagggcctgggacctgaagcagctgcaggtccaagagcgccccaaggccttgcaggctgaggtggccctgaccctgaaggtctgggagaacatgactgactcagccctggccaccatcctgggccagcctcttcatacactgagccacattcactcccagctgcagacctgtacacagcttcaggccacagcagagcccaggtccccgagccgccgcctctcccgctggctgcacaggctccaggaggcccagagcaaggagacccctggctgcctggaggcctctgtcacctccaacctgtttcgcctgctcacccgggacctcaagtgtgtggccaatggagaccagtgtgtctg

SEQ ID NO: 46 - DNA, 582 bp, Mus musculus
  source 1..582: mol_type = other DNA
atgctcctcctgctgttgcctctgctgctggccgcagtgctgacaagaacccaagctgaccctgtccccagggccaccaggctcccagtggaagcaaaggattgccacattgctcagttcaagtctctgtccccaaaagagctgcaggccttcaaaaaggccaagggtgccatcgagaagaggctgcttgagaaggacatgaggtgcagttcccacctcatctccagggcctgggacctgaagcagctgcaggtccaagagcgccccaaggccttgcaggctgaggtggccctgaccctgaaggtctgggagaacataaatgactcagccctgaccaccatcctgggccagcctcttcatacactgagccacattcactcccagctgcagacctgtacacagcttcaggccacagcagagcccaagcccccgagtcgccgcctctcccgctggctgcacaggctccaggaggcccagagcaaggagactcctggctgcctggaggactctgtcacctccaacctgtttcaactgctcctccgggacctcaagtgtgtggccagtggagaccagtgtgtctga